(19) United States
(12) Patent Application Publication
(10) Pub. No.: US 2020/0146881 A1
(43) Pub. Date: May 14, 2020

(54) MOLDABLE HEATER WITH MINIATURE HARMONIC RESONANCE FREQUENCY VIBRATION GENERATOR FOR OPTHALMIC EYELID THERAPY INCLUDING NEUROSTIMULATION DEVICE

(71) Applicant: EYEDETEC MEDICAL, INC., Danville, CA (US)

(72) Inventors: Barry J. Linder, Danville, CA (US); Ronald D. Linder, Gilbert, AZ (US); James L. Dippo, Arvada, CO (US); Rubin D. Linder, Danville, CA (US); John A. Scholl, San Ramon, CA (US); Vance Thompson, Sioux Falls, SD (US)

(73) Assignee: EYEDETEC MEDICAL, INC., Danville, CA (US)

(21) Appl. No.: 16/739,958

(22) Filed: Jan. 10, 2020

Related U.S. Application Data
(63) Continuation-in-part of application No. 16/146,396, filed on Sep. 28, 2018.
Application No. 16/146,396 is a continuation-in-part of application No. 15/187,457, filed on Jun. 20, 2016.
(60) Provisional application No. 62/565,818, filed on Sep. 29, 2017.
(60) Provisional application No. 62/230,843, filed on Jun. 18, 2015.

(51) Int. Cl. A61F 7/02; A61H 1/00

(52) U.S. Cl. CPC A61F 7/02; A61H 1/00; A61F 2007/0004; A61H 2201/0207; A61H 2205/024

(57) ABSTRACT

Embodiments provide a moldable warming device, including a heating disc; a first harmonic resonance frequency stimulation vibration generator (RFSVG); a coupling device; a mask configured to hold the heating disc, the first harmonic RFSVG, and the coupling device for use in parallel utility; and a sensor array, the sensor array configured to determine tuning parameters of a vibration and heating profile of a user's individual eyelid, periorbital, and nasal three-dimensional anatomy and surface topography. According to at least one embodiment, the moldable warming device is configured to provide an eyelid surface and periorbital structures with therapeutic warmth, and the first harmonic RFSVG is configured to provide tuned harmonic resonance or non-resonance frequency stimulation vibration over the user's nasal bridge area through the user's nasal bone such that nasal sensory nerves are stimulated to induce tear production reflex in the user's lacrimal function unit.

### CROSS REFERENCE TO RELATED APPLICATIONS

This application is a continuation-in-part application of U.S. patent application Ser. No. 15/187,457, filed on Jun. 20, 2016, entitled “MOLDABLE HEATER WITH MINIATURE HARMONIC RESONANCE FREQUENCY VIBRATION GENERATOR FOR OPHTHALMIC EYELID THERAPY,” which claims priority to U.S. Provisional Patent Application Ser. No. 62/230,843, filed on Jun. 18, 2015; this application is a continuation-in-part application of U.S. patent application Ser. No. 16/146,396, filed on Sep. 28, 2018, entitled “MOLDABLE HEATER WITH MINIATURE HARMONIC RESONANCE FREQUENCY VIBRATION GENERATOR FOR OPHTHALMIC EYELID THERAPY,” which claims priority to U.S. Provisional Patent Application Ser. No. 62/565,818, filed on Sep. 29, 2017; U.S. patent application Ser. No. 16/146,396 is a continuation-in-part application of the aforementioned U.S. patent application Ser. No. 15/187,457, filed on Jun. 20, 2016, which claims priority to the aforementioned U.S. Provisional Patent Application Ser. No. 62/230,843, filed on Jun. 18, 2015; all of the above-referenced applications are hereby incorporated by reference in their entireties into this application.

### BACKGROUND

### Field

Embodiments relate generally to devices and methods for treating and monitoring dry eye disease. More particularly, embodiments of the disclosure relate to wearable eye masks that include heating elements and resonance frequency stimulation vibration generators for providing mobilization of meibum within the eyelid's Meibomian Glands, and neurostimulation in the nasal sensory area to effect neuromodulation of tear production including Meibomian Gland expression.

### Description of the Related Art

Eye patch devices are known in the art. For example, U.S. Pat. No. 4,682,371 describes a protective eye patch. The '371 patch has several tabs for securing the patch to a patient's eye. U.S. Pat. No. 3,068,863 describes a patch designed to keep the eye closed. U.S. Pat. No. 3,092,103 describes a patch with a cushion material at the edge that allows the patient's eye to move underneath the eye patch. U.S. Pat. No. 3,908,645 describes an ophthalmic therapeutic pressure bandage with a conformable, permeable carrier tape.

U.S. Pat. No. 6,409,746 describes an eye pillow that releases steam from its surface applied to the eyes and the area around the eyes. The temperature described in the '746 patent is 50° C. or lower and has a total weight of 50 g or more.

Several conditions exist for which medical and cosmetic therapy is appropriate. For example, blepharitis, meibomitis, chalazia, and/or styes are common disorders of the eyelids that cause chronic inflammation in the peri-orbita, and are often associated with ocular tear film abnormalities resulting in dry eye disease and symptoms. Symptoms of dry eye disease and blepharitis include burning, itching, light sensitivity, blurred vision, tearing, and foreign body sensation. Signs include eyelash crusting, ocular discharge, eyelid scaling and swelling, corneal staining, and conjunctival redness. For example, staphylcoccal blepharatis is often associated with scaling and crusting along the eye lashes. There is no cure for dry eyes, and long-term treatment is required to keep it under control.

The predominant cause of dry eye is an insufficient or abnormal lipid layer of the surface of the tear film. In a healthy eye, this oily layer inhibits the evaporation of the water based sub layers of the tear film, thereby maintaining a stable tear film. These lipids are produced in the Meibomian glands located in the eyelids. From about 24 to about 40 Meibomian glands exist in each eyelid. For those suffering evaporative dry eye disease, the likely root cause is Meibomian glands that have become filled with viscous lipids, and occasionally clogged, resulting in a reduced quantity and abnormal quality of lipids flowing out onto the tear film. Meibomitis, also known as Meibomian Gland Dysfunction (MGD), is a dysfunction of the Meibomian gland and limits the gland's ability to provide a normal lipid-based oily layer as a critical component of the eye's natural tear film.

Currently available treatments for dry eye disease and related conditions include warm compresses for 5-15 minutes, such as a warm washcloth, that heats the debris and crust on the lid, and lowers the viscosity of the lipids in the Meibomian glands. After the lid has been warmed, occasionally a lid scrub is performed by using a suitable soap, such as Neutrogena® or Johnson's Baby Shampoo®. Commercially available cleansing pads are available to assist in performing the lid scrub, for example OCuSOFT® Lid Scrubs or Novartis Ophthalmics Eye Scrub®. Following the eye scrub, antibiotics, such as polysporin, tobramycin, or erythromycin can be applied, to alleviate patient discomfort and reinforce the treatment.

Warm moist compress therapy applied to the skin of the closed eyelids increases tear-film lipid layer thickness for subjects with MGD by more than 80% after 5 minutes of initiating treatment and an additional 20% after 15 minutes of treatment. The transition temperature from a solid to a liquid for Meibomian lipids is actually a range from 28° C. to 32° C. because of differences between individual's mixture of lipids. The temperature of the eyelids will therefore affect the liquidity of Meibomian lipids and hence their viscosity. The non-Newtonian lipid mixture is known to undergo shear thinning when exposed to shear forces. Further it is known that oscillations enhance the flow rate of a shear-thinning fluid.

Conventional ocular heating devices, such as warm compresses, typically require an external power source. These sources include electricity, a stove top boiling preparation, or a microwave appliance, and are consequently difficult to provide a controlled temperature to the eyelids, are labor intensive, cumbersome, and inconvenient, and therefore historically result in poor patient compliance and persistence with the recommended therapy. Some success is realized with in-office, doctor-assisted visits.

What is needed is a convenient, accurate, and effective, easily used hand moldable heating source that patients or their doctors apply via a coupling mechanism to patient's eyelids, and which delivers a therapeutic temperature to the entire eyelid surface independent of the individual's orbital anatomy, for a sufficient length of time to be effective. There is also a need for a device or component of the system that incorporates a moldable material to serve as a coupling element, heated and able to deliver heat and resonance frequency stimulation vibration to the target tissue of the eyelid, as well as to detect a positive eyelid resonant response from a broad range of generated harmonic frequencies, thus allowing a personalized or custom approach to each individual user. There is also a need for a device or component of the system that incorporates a neurostimulation unit to deliver resonance or non-resonance frequency stimulation vibration to the sensory nerves in the nasal area to induce neuromodulation of the tear production reflex including Meibomian Gland expression.

### SUMMARY

Embodiments relate to a reusable system and one-time use or reusable components for a heating device with a reusable miniature harmonic frequency stimulation generator inside a reusable eye mask with a reusable or one-time use coupling device. The system according to various embodiments ensures proper delivery of targeted heat therapy and appropriately tuned harmonic resonance frequency stimulation to the location of the eyelids, from the inner surface of the eyelid or over their entire eyelid external skin surface. The system according to various embodiments mobilized Meibom lipids within the Meibomian glands, unblocks clogged Meibomian glands and ducts, lowers viscosity of Meibom, and improves flow of Meibom lipid to the surface of the eye and onto the tear film.

Embodiments provide a moldable heating device having a flexible and moldable three-dimensional shape which allows desired, uniform heat transfer to the area stated while the composition of the heater controls the temperature and desired length of time heat is generated and delivered to the eyelid. The reusable miniature resonance frequency stimulation vibration generator is set to a specific patient frequency stimulation to encourage the production and flow of natural tear lipid component from excitation of the Meibomian glands in the eyelids. In addition to warming the eyelid surface and Meibomian glands directly, the heat to the surrounding periorbital area increases vascular perfusion and thereby naturally increases tissue temperature. Thusly heated, the viscosity of the lipid in the Meibomian glands is decreased.

According to at least one embodiment, the addition of harmonic resonance frequency stimulation generation and applied shear forces tuned to the patient's harmonic resonance of their eyelids and Meibomian glands encourages flow of the lipids expressed in these glands, delivering this critical tear film lipid layer onto the ocular surface.

According to another embodiment, there is provided an externally powered heater in the mask along with the externally powered harmonic resonance frequency stimulation generator. The external energy source allows for circuitry in an external power module to act as a sweep range device, targeting the specific optimal point or sweep frequency stimulation to be tuned to the individual patient's eyelids during initial testing by the doctor. The initial testing device would be supplied to the doctor and would be a device with sensing elements to identify the optimally tuned harmonic resonance frequency stimulation. Once the frequency stimulation is defined and treatment provided in the doctor's office, the patient would optionally receive a reusable mask with custom harmonic resonance frequency stimulation vibration generators set to match doctor's test optimized treatment parameters. This configuration would be amenable to both single use and reusable heating warming wafer disc approaches.

According to at least one embodiment, there is provided a moldable warming device with a miniature harmonic resonance frequency stimulation vibration generator. The moldable warming device includes a moldable heating disc; the miniature harmonic resonance frequency stimulation vibration generator; a moldable coupling device; a reusable mask configured to hold the moldable heating disc, the miniature harmonic resonance frequency stimulation generator, and the moldable coupling device for use in parallel utility; and a sensor array configured to determine tuning parameters of a vibration and heating profile of a user's individual patient eyelid and periorbital three-dimensional anatomy and surface topography.

According to at least one embodiment, the moldable heating disc is comprised of a polymer, resulting in a pliable and strong moldable material having high shape retention characteristics.

According to at least one embodiment, the moldable heating disc includes a single-use heating disc, wherein the polymer is manufactured to provide the moldable heating disc with a 20-80% pore volume, resulting in high porosity in the moldable heating disc.

According to at least one embodiment, the moldable heating disc is infused with FeOₓ heating material.

According to at least one embodiment, the FeOₓ heating material has a reaction 4Fe(s)+3O₂(g)→2Fe₂O₃(s) and is comprised of a ratio of ingredients and produces a calibrated heater temperature at the eyelid surface for the time duration of about 5-15 minutes necessary for optimal patient therapeutic effect.

According to at least one embodiment, the moldable heating disc includes a defined pocket indentation for locating the miniature harmonic resonance frequency stimulation vibration generator.

According to at least one embodiment, the miniature harmonic resonance frequency stimulation vibration generator is preset to a defined harmonic resonance frequency stimulation for optimal patient therapeutic effect.

According to at least one embodiment, the preset harmonic resonance frequency stimulation includes frequencies of about 2 Hz-270 Hz, about 15 Hz-40 Hz, or about 30 Hz-60 Hz.

According to at least one embodiment, the preset harmonic resonance frequency stimulation for each patient is determined by a health care provider.

According to at least one embodiment, the preset harmonic resonance frequency stimulation is determined using an in-office, variable harmonic resonance frequency stimulation vibration generator configured to test available resonance frequencies to identify the individual patient's optimal therapeutic frequency stimulation for tear film stabilization and flow of Meibom from the patient's Meibomian glands.

According to at least one embodiment, the moldable warming device further includes a miniature, ultrasensitive biochemical sensor configured to detect an optimal tissue response to the preset harmonic resonance frequency stimulation.

According to at least one embodiment, the moldable warming device further includes a miniature, ultrasensitive biochemical sensor configured to map a biochemical makeup of the user's Meibomian lipids or glands.

According to at least one embodiment, the coupling device includes one of an eye patch or a reusable mask.

According to at least one embodiment, the coupling device includes one of breathable cotton, linen, bamboo, or hemp.

According to at least one embodiment, the coupling device is configured to hold the miniature resonance frequency stimulation vibration generator.

According to at least one embodiment, the coupling device includes one or two pockets for holding the moldable heating disc without the miniature resonance frequency stimulation vibration generator.

According to at least one embodiment, the coupling device is further comprised of a vibration transfer facilitating material, such as metal wire woven into one of the breathable cotton, linen, bamboo, or hemp.

According to at least one embodiment, the coupling device further includes an electrical lead with a connector configured to drive both the miniature resonance frequency stimulation vibration generator and the moldable heating disc.

According to at least one embodiment, the moldable heating disc includes nan-particles of ferrous or non-ferrous metals to facilitate both heat transfer and harmonic resonance frequency stimulation transfer to the user's Meibomian glands.

According to at least one embodiment, the moldable heating disc includes nanoparticles of ferrous or non-ferrous metals and nano-ceramic particles to facilitate heat transfer, resonance frequency stimulation vibration transfer, and control a heat transfer rate to the user's Meibomian glands.

According to at least one embodiment, the coupling device includes a coupling agent material including hydrogel used for transferring heat and harmonic stimulation vibration energy or movement to the eyelid surface and the user's Meibomian glands.

According to at least one embodiment, the coupling agent material transmits the mechanical response from the eyelid surface and the user's Meibomian glands to a sensor.

According to at least one embodiment, the coupling agent material is a single use, disposable sterile or non-sterile component.

According to at least one embodiment, the coupling device further includes a solution sensor integrated into the coupling device, wherein the solution sensor is configured to record a response.

According to at least one embodiment, the coupling device further includes an integrated piezoelectric device configured to record the response and compare the response to a patient subjective impression of predetermined frequency stimulation.

Embodiments provide a moldable warming device. The moldable warming device includes a heating disc, a harmonic resonance frequency stimulation vibration generator (RFSVG), a coupling device, a mask, and a sensor array. The mask is configured to hold the heating disc, the harmonic RFSVG, and the coupling device for use in parallel utility. The sensor array is configured to determine tuning parameters of a vibration and heating profile of a user's individual eyelid and periorbital three-dimensional anatomy and surface topography. The moldable warming device is configured to provide an entire eyelid surface and periorbital structures with therapeutic warmth and tuned harmonic resonance frequency stimulation vibration to mobilize Meibom lipids and stimulate flow of the mobilized Meibom lipids from the user's Meibomian glands.

According to at least one embodiment, the mask includes a shape memory bridge allowing the user flexibility forming the mask to the user's individual eyelid and periorbital three-dimensional anatomy and surface topography.

According to at least one embodiment, the coupling device is configured to contact the user's eyelid skin for heat and vibration transfer.

According to at least one embodiment, the mask includes a heat reflective material configured to reflect and direct heat from the heating disc toward the user's eyelid surface.

According to at least one embodiment, the sensor array includes a thermocouple configured to record temperature of the user's eyelid surface. In some embodiments, the sensor array includes a flexible printed circuit board where the thermocouple is embedded therein. In some embodiments, the flexible printed circuit board comprises a polyimide film.

According to at least one embodiment, the sensor array includes temperature sensors, pressure sensors, moisture sensors, pH sensors, and combinations thereof.

According to at least one embodiment, the moldable warming device further includes a vibration modulation controller. The vibration modulation controller controls the stimulation of the harmonic RFSVG.

According to at least one embodiment, the moldable warming device further includes a microprocessor and a wireless transmitter. The microprocessor converts analog data generated from the sensor array into a digital data stream. The wireless transmitter transmits the digital data stream wirelessly via an antenna. In some embodiments, the wirelessly transmitted digital data stream is configured to be received by a smart device. In some embodiments, the wirelessly received digital data stream is configured to be processed by a smart device application.

Embodiments provide a method for ophthalmic eyelid therapy. The method includes the steps of applying a moldable warming device to a user's individual eyelid and periorbital three-dimensional anatomy and surface topography, generating thermal energy and harmonic resonance frequency stimulation vibration, transferring the thermal energy and the harmonic resonance frequency stimulation vibration to either the inner surface of the eyelid or an entire eyelid external skin surface and periorbital structures, and mobilizing Meibom lipids and stimulating flow of the mobilized Meibom lipids from the user's Meibomian glands. The moldable warming device includes a heating disc, a harmonic RFSVG, a coupling device, a mask, and a sensor array. The mask is configured to hold the heating disc, the harmonic RFSVG, and the coupling device for use in parallel utility.

According to at least one embodiment, the transferring step includes transferring the thermal energy and the harmonic resonance frequency stimulation vibration to an inner surface of the eyelid.

According to at least one embodiment, the mask includes a shape memory bridge allowing the user flexibility forming the mask to the user's individual eyelid and periorbital three-dimensional anatomy and surface topography.

According to at least one embodiment, the coupling device is configured to contact the user's eyelid skin for heat and vibration transfer.

According to at least one embodiment, the mask includes a heat reflective material configured to reflect and direct heat from the heating disc toward the user's eyelid surface.

According to at least one embodiment, the sensor array includes a thermocouple configured to record temperature of the user's eyelid surface. In some embodiments, the sensor array includes a flexible printed circuit board where the thermocouple is embedded therein.

According to at least one embodiment, the sensor array includes temperature sensors, pressure sensors, moisture sensors, pH sensors, and combinations thereof.

According to at least one embodiment, the method further includes the step of generating, at the sensor array, a data stream responsive to a vibration and heating profile of the user's individual patient eyelid and periorbital three-dimensional anatomy and surface topography. In some embodiments, the method further includes the steps of converting the data stream from analog to digital, and transmitting the data stream wirelessly via an antenna. In some embodiments, the method further includes the step of receiving the wirelessly transmitted data stream using a smart device. In some embodiments, the method further includes the step of processing the wirelessly transmitted data stream using a smart device application.

Embodiments provide a moldable warming device. The moldable warming device includes a heating disc, a first harmonic RFSVG, a coupling device, a mask, and a sensor array. The mask is configured hold the heating disc, the first harmonic RFSVG, and the coupling device for use in parallel utility. The sensor array is configured to determine tuning parameters of a vibration and heating profile of a user's individual eyelid, periorbital, and nasal three-dimensional anatomy and surface topography. The moldable warming device is configured to provide an eyelid surface and periorbital structures with therapeutic warmth. The first harmonic RFSVG is configured to provide tuned harmonic resonance or non-resonance frequency stimulation vibration over the user's nasal bridge area through the user's nasal bone such that nasal sensory nerves are stimulated to induce tear production reflex in the user's lacrimal function unit.

According to at least one embodiment, the moldable warming device further includes a second harmonic RFSVG. The mask is configured to hold the second harmonic RFSVG. The second harmonic RFSVG is configured to provide the eyelid surface and periorbital structures with tuned harmonic resonance or non-resonance frequency stimulation vibration.

According to at least one embodiment, the coupling device is configured to contact the user's eyelid skin for heat transfer.

According to at least one embodiment, the coupling device includes hydrogel.

According to at least one embodiment, the mask includes a heat reflective material configured to reflect and direct heat from the heating disc toward the user's eyelid surface.

According to at least one embodiment, the sensor array includes a thermocouple configured to record temperature of the user's eyelid surface. According to at least one embodiment, the sensor array includes a flexible printed circuit board where the thermocouple is embedded therein. According to at least one embodiment, the flexible printed circuit board includes a polyimide film.

According to at least one embodiment, the sensor array includes sensors such as temperature sensors, pressure sensors, moisture sensors, pH sensors, and combinations thereof.

According to at least one embodiment, the moldable warming device further includes a vibration modulation controller. The vibration modulation controller controls the stimulation of the first harmonic RFSVG.

According to at least one embodiment, the moldable warming device further includes a microprocessor and a wireless transmitter. The microprocessor converts analog data generated from the sensor array into a digital data stream. The wireless transmitter transmits the digital data stream wirelessly via an antenna. According to at least one embodiment, the wirelessly transmitted digital data stream is configured to be received by a smart device. According to at least one embodiment, the wirelessly received digital data stream is configured to be processed by a smart device application.

Embodiments provide a method for ophthalmic eyelid therapy. The method includes the step of applying a moldable warming device to a user's individual eyelid, periorbital, and nasal three-dimensional anatomy and surface topography. The moldable warming device includes a heating disc, a first harmonic RFSVG, a coupling device, a mask, and a sensor array. The mask is configured to hold the heating disc, the first harmonic RFSVG, and the coupling device for use in parallel utility. The method includes the step of generating thermal energy and transferring the thermal energy to an eyelid surface and periorbital structures. The method includes the step of generating harmonic resonance or non-resonance frequency stimulation vibration and transferring the harmonic resonance or non-resonance frequency stimulation vibration over the user's nasal bridge area through the user's nasal bone. The method includes the step of stimulating nasal sensory nerves to induce tear production reflex in the user's lacrimal function unit.

According to at least one embodiment, the thermal energy is transferred to an inner surface of the eyelid.

According to at least one embodiment, the coupling device is configured to contact the user's eyelid skin for heat transfer.

According to at least one embodiment, the coupling device includes hydrogel.

According to at least one embodiment, the mask includes a heat reflective material configured to reflect and direct heat from the heating disc toward the user's eyelid surface.

According to at least one embodiment, the sensor array includes a thermocouple configured to record temperature of the user's eyelid surface. According to at least one embodiment, the sensor array includes a flexible printed circuit board where the thermocouple is embedded therein.

According to at least one embodiment, the sensor array includes sensors such as temperature sensors, pressure sensors, moisture sensors, pH sensors, and combinations thereof.

According to at least one embodiment, the method further includes the step of generating, at the sensor array, a data stream responsive to a vibration and heating profile of the user's individual patient eyelid and periorbital three-dimensional anatomy and surface topography. According to at least one embodiment, the method further includes the step of converting the data stream from analog to digital. The method further includes the step of transmitting the data stream wirelessly via an antenna. According to at least one embodiment, the method further includes the step of receiving the wirelessly transmitted data stream using a smart device. According to at least one embodiment, the method further includes the step of processing the wirelessly transmitted data stream using a smart device application.

Embodiments provide a moldable warming device. The moldable warming device includes a heating disc, a first harmonic RFSVG, a coupling device, and a mask. The mask is configured to hold the heating disc, the first harmonic RFSVG, and the coupling device for use in parallel utility. The moldable warming device is configured to provide an eyelid surface and periorbital structures with therapeutic warmth. The first harmonic RFSVG is configured to provide tuned harmonic resonance or non-resonance frequency stimulation vibration over the user's nasal bridge area through the user's nasal bone such that nasal sensory nerves are stimulated to induce tear production reflex in the user's lacrimal function unit.

According to at least one embodiment, the moldable warming device further includes a second harmonic RFSVG. The mask is configured to hold the second harmonic RFSVG. The second harmonic RFSVG is configured to provide the eyelid surface and periorbital structures with tuned harmonic resonance or non-resonance frequency stimulation vibration.

Embodiments provide a method for ophthalmic eyelid therapy. The method includes the step of applying a moldable warming device to a user's individual eyelid, periorbital, and nasal three-dimensional anatomy and surface topography. The moldable warming device includes a heating disc, a first harmonic RFSVG, a second harmonic RFSVG, a coupling device, and a mask. The mask is configured to hold the heating disc, the first harmonic RFSVG, the second harmonic RFSVG, and the coupling device for use in parallel utility. The method includes the step of reducing viscosity of the user's meibum by generating thermal energy via the heating disc and transferring the thermal energy to the user's eyelid surface and periorbital structures. The method includes the step of mobilizing the user's meibum by generating harmonic resonance or non-resonance frequency stimulation vibration via the first harmonic RFSVG and transferring the harmonic resonance or non-resonance frequency stimulation vibration to the user's eyelid surface and periorbital structures in the absence of transferring the thermal energy to the user's eyelid surface and periorbital structures. The method includes the step of stimulating the user's nasal sensory nerves to induce tear production reflex in the user's lacrimal function unit by generating the harmonic resonance or non-resonance frequency stimulation vibration via the second harmonic RFSVG and transferring the harmonic resonance or non-resonance frequency stimulation vibration over the user's nasal bridge area through the user's nasal bone nasal bridge area.

### BRIEF DESCRIPTION OF THE DRAWINGS

So that the manner in which the recited features, aspects and advantages of the disclosure, as well as others that will become apparent, are attained and can be understood in detail, a more particular description of certain embodiments briefly summarized above can be had by reference to the embodiments that are illustrated in the drawings that form a part of this specification. It is to be noted, however, that the appended drawings illustrate only exemplary embodiments and are, therefore, not to be considered limiting of the disclosure's scope, for the disclosure can admit to other equally effective embodiments. Like numbers refer to like elements throughout, and the prime notation, if used, indicates similar elements in alternative embodiments or positions.

FIG. 1A is a front view of a heating disc device; FIG. 1B is a side or section view of the heating disc.

FIGS. 2A-2F are graphs of the force velocity and differential pressure for various porous polyolefin compositions.

FIG. 3A is a front view of a packaged heating disc; FIG. 3B is a section view of a packaged heating disc.

FIG. 4A and FIG. 4B are a side section view and front section view heater disk showing embedded metallic nanoparticles.

FIG. 5A and FIG. 5B are a side section view and front section view heater disk showing embedded metallic and ceramic nanoparticles.

FIG. 6A is a front view of the heating disc with attached resonance frequency stimulation generator; FIG. 6B is a side view of the heating disc with attached resonance frequency stimulation generator and USB connector.

FIG. 7A is a reusable mask incorporating both a miniature resonance frequency stimulation vibration generator and permanent imbedded heater element; FIG. 7B shows an example vibration modulation controller; FIG. 7C shows an example mechanical resonator; FIG. 7D shows an example control board.

FIG. 8 is a representative far infrared front-end spot heater.

FIG. 9A is a section view of a microfluidic sensor; FIG. 9B is a front view of a microfluidic sensor.

FIG. 10A is a schematic for a dual channel solution sensing electrode; FIG. 10B is a schematic for a single channel solution sensing electrode.

FIG. 11 is a photograph of the mask prototype with the proposed 3D shape memory element.

FIG. 12A is a front view of the EyeGiene® heating device demonstrating temperature and frequency stimulation generator possible mask locations, along with in-place signal conditioner and amplifying circuitry. FIG. 12B is a top view of the original moldable heater with the new addition of a miniature resonance frequency stimulation generator located in the new pocket which is formed when the moldable heater is manufactured. The frequency stimulation generator is a reusable electrical device, which snaps into place for the duration of the one-time use or reusable heater element and is then removed before the one-time use or reusable heater element is removed. This view shows the heat shield material incorporated into the design for thermal manipulation of heater element, energy output.

FIG. 13 further demonstrates the frequency stimulation controller with a temperature controller incorporated into the unit.

FIG. 14 is an electrical schematic demonstrating the circuitry to localize and transmit sensor information via antenna to a smartphone or smart device receiver, processor, and display.

FIG. 15 is a block diagram of the path to transmit data to a smartphone or smart device receiver, processor, and display.

FIG. 16 further shows a block diagram showing a smartphone or smart device as the receiver, processor, and display.

FIG. 17A is a front view drawing of a single location temperature sensor and frequency stimulation generator. The thermocouples have wireless transmission capability and the power is provided by a localized power source, replaceable battery, or USB. FIG. 17B further shows a top view of the single point sensor showing the sensor on the eyelid side of the mask.

FIGS. 18A-18C are photographs depicting a single temperature sensor with wireless transmission capabilities.

FIG. 19 is a drawing of the front view of a multiple temperature sensor array using a flexible printed circuit for eye shape capabilities and data collection via wireless transmission to external components such as a smartphone or smart device, wireless receiver, USB, processor, or display.

FIGS. 20A and 20B show photographs of a flexible printed circuit with temperature sensors or sensor array.

FIG. 21 shows a mask with multiple sensor capability. The sensor array detects temperature, pressure, moisture, pH, and frequency stimulation. This array communicates via wireless technology to a smartphone or smart device.

FIG. 22A shows a photograph of a miniaturized temperature sensing device. FIG. 22B shows a performance graph of the miniaturized temperature sensing device. FIG. 22C shows a depiction of a miniaturized moisture/humidity sensing device. FIGS. 22D-1 and 22D-2 shows photographs of a miniaturized pressure sensing device. FIG. 22E shows a depiction of a miniaturized pH sensing device. FIG. 22F shows a photograph of the miniaturized pH sensing device.

FIG. 23 is a commercial wire schematic for a smartphone or smart device to receive process, and display data from the wireless sensor array.

FIG. 24A is a graphical representation showing a front view of a human eye. FIG. 24B is a graphical representation showing an enlarged cross-sectional view of an upper eyelid of the human eye. FIG. 24C is a graphical representation showing a cross-sectional view through a Meibomian gland of an upper eyelid of a human eye where the harmonic resonance frequency stimulation vibration mobilizes and stimulates flow of the Meibom lipids within a Meibomian gland. The mobilized and stimulated flow leads to enhanced excretion of the Meibom lipids.

FIG. 25 is a front view drawing of a heating mask including a neurostimulation unit for providing resonance or non-resonance frequency stimulation vibration to the nasal sensory nerves for inducing through controlled neuromodulation a tear production reflex including Meibomian Gland expression.

### DETAILED DESCRIPTION

While the scope of the system and method will be described with several embodiments, it is understood that one of ordinary skill in the relevant art will appreciate that many examples, variations and alterations to the systems and methods described here are within the scope and spirit of the embodiments.

Accordingly, the embodiments described are set forth without any loss of generality, and without imposing limitations, on the embodiments. Those of skill in the art understand that the scope includes all possible combinations and uses of particular features described in the specification.

As used throughout, one of ordinary skill in the relevant art would understand that the following terms can all be used interchangeably: dry eyes, dry eye disease, dry eye syndrome, evaporative dry eye, lipid deficiency dry eyes, blepharitis, Meibomian gland disease, Meibomian gland dysfunction, and MGD.

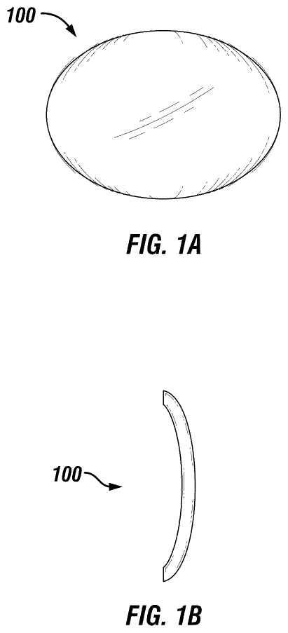

Referring now to the disclosed devices in more detail, in FIG. 1A and FIG. 1B, there is shown a heating disc 100 manufactured to fit comfortably in a reusable mask conforming to the natural shape of the closed eyelid surface, and surrounding periorbital area. A suitable shape is an oval configuration better describing the shape of the ocular surface, but can also be manufactured in rectangle or square shapes that are bigger than this minimum oval requirement.

In further detail, still referring to the devices disclosed in FIG. 1A and FIG. 1B, the heating disc 100 shows a side view of an initial curvature provided to conform the device to the closed eyelid surface by the patient when in use. The device is manufacturable such that it has a first configuration, such as an initial curvature as shown, and is conformable by the patient, physician or technician into a second configuration when in use. The material is pliable and can be easily shaped to maximize the contact area between the mask and the closed eyelid surface, to accurately match an individual user's particular orbital anatomy. For example, there will be a difference in shape for a user with a deeply set eye and deep orbit versus a user with an anteriorly placed globe. The heating disc 100 can be shaped by hand to fit a particular orbital anatomy. This approach will accurately match an individual user's particular orbital anatomy with the heating disc's high shape retention characteristics. Alternatively, light compression from the reusable eye mask will conform the heating disc 100 to the surface of the closed eyelids.

The desired heating disc integrity and heating duration are achieved by controlling disc thickness, formulation of the heating material, and porosity that allows controlled air flow to the heating material. Ideal time for application of heat is in a range from about 5 to 30 minutes, preferably from about 5 to 15 minutes and temperature at the surface of the eyelid should be between about 40 and 46° C. Example polyethylene (PE) based materials with a usable 25-60 μm pore size (PE25 through PE60) are shown in Table 1.

A nominal pore volume of 50% will allow the heating disc 100 to be reshaped or molded by the patient. The porous and moldable PE based material can have nominal pore sizes of 7-150 μm and are manufactured up to 300 μm in pore size. Another polyolefin material, polypropylene (PP), (PP-100 and PP150), shown in Table 1, is a heating disc material with 100-150 μm pore size with a smaller 45% pore volume, and can be infused with larger heater material particles for a longer disc heating time of 20-25 minutes.

TABLE 1
Porous plastic and moldable materials

| Material Parameter | PE10 | PE25 | PE60 | PE100 | PE125 | PE-HV | PP50 | PP100 | PP150 | PVDF30 | PTFE30 |
|---|---|---|---|---|---|---|---|---|---|---|---|
| Polymer Type | PE | PE | PE | PE | PE | PE | PP | PP | PP | PVDF | PTFE |
| Material Options | H2O/SS | H2O/SS | H2O/SS | H2O/SS | H2O/SS | H2O/SS | H2O/SS | H2O/SS | H2O/SS | | |
| Nominal Pore Size (μ) | 10 | 25 | 60 | 100 | 150 | 30 | 50 | 100 | 150 | 30 | 30 |
| Nominal Pore Volume (%) | 50 | 50 | 50 | 50 | 50 | 60 | 45 | 45 | 45 | 45 | 40 |
| Air Permeability | <10 | 10-50 | 30-90 | 50-100 | 70-120 | 40-80 | 40-120 | 60-150 | 70-200 | 10-50 | 5-25 |
| (ft/min @ 1.2″ H2O ΔP, | | | | | | | | | | | |
| material thickness of .125″) | | | | | | | | | | | |
| H2O Intrusion Pressure | 175 | 152 | 70 | 42 | 16 | 65 | 35 | 12 | 10 | 60 | 80 |
| (mBar) | | | | | | | | | | | |
| Air Filtration | .5μ | 1μ | 5μ | 15μ | 30μ | 1μ | 10μ | 50μ | 75μ | 3μ | 3μ |
| (ε > 98% @ 50 ft/min) | | | | | | | | | | | |
| Water Filtration | 5μ | 10μ | 15μ | 20μ | 90μ | 10μ | 40μ | 100μ | 150μ | 15μ | |
| (ε > 95% @ 4 ft/min) | | | | | | | | | | | |
| Relative Raw Material | $$$$ | $ | $ | $ | $ | $$ | $$ | $$ | $$ | $$$ | $$$$ |
| Price | | | | | | | | | | | |

The heating disc 100 may be made of a broad combination of the ingredients resulting in a sufficiently rigid and strong molded material that can hold its shape, yet is easily hand moldable to the closed eyelid surface for optimal therapeutic effect. The material porosity allows a heating material to reside in the pathways with access to air at between 10-90 ft/min @ 1.2″ H₂O ΔP, where the material is between 0.125″ (3.175 mm) and 0.250″ (6.35 mm) thick with enough porosity space to adhere sufficient heating material to the support surface and internal sites. Further, the various ingredients of the disc can be substituted for different materials by shape and size to control the heating rate, total thermal energy converted and delivered, and longevity of the heat conversion.

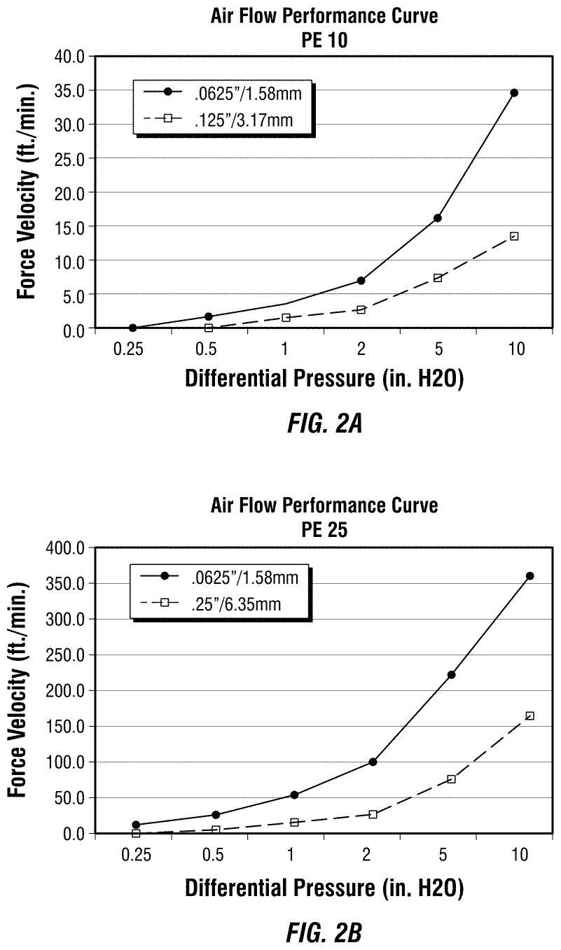

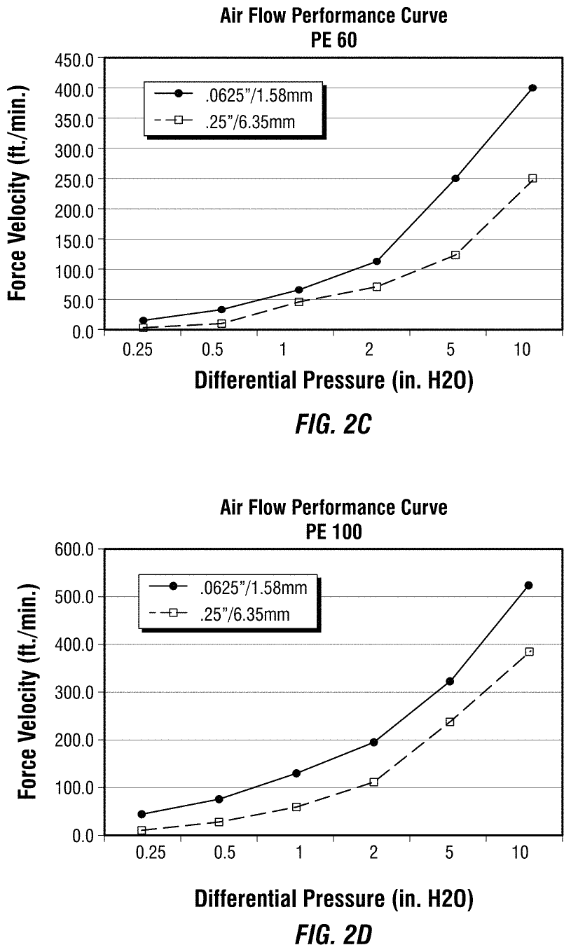

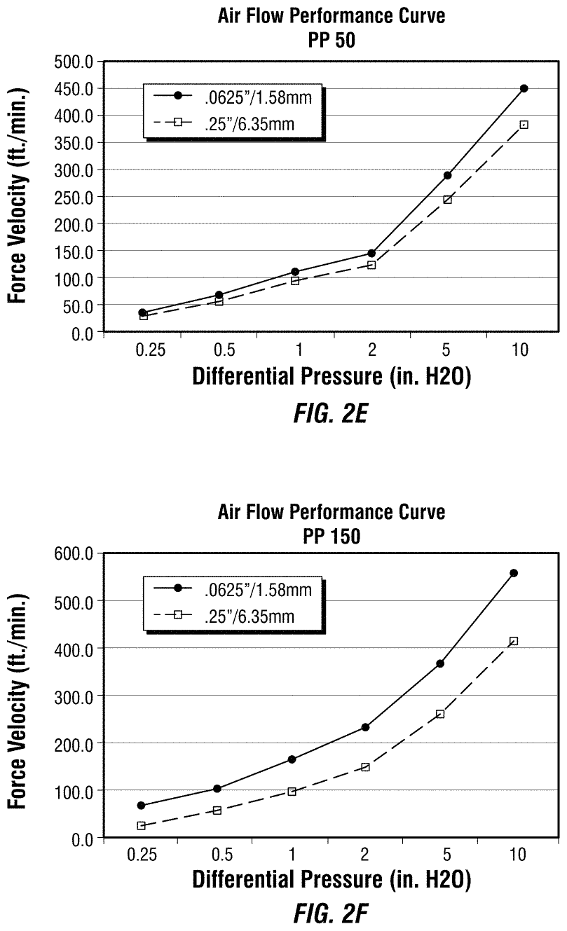

FIGS. 2A-F are graphs of the force velocity and differential pressure for various porous polyolefin compositions. Porous polyolefin compositions are used for the malleable heater disc support material described herein. FIG. 2A shows the air flow performance curve of force velocity (ft/min) and differential pressure (inches H₂O) with a thickness of 0.0625″ and 0.125″ for the PE10 material shown in Table 1. FIG. 2B shows the air flow performance curve of force velocity and differential pressure with a thickness of 0.0625″ and 0.25″ for the PE25 material shown in Table 1. FIG. 2C shows the air flow performance curve of force velocity and differential pressure with a thickness of 0.0625″ and 0.25″ for the PE60 material shown in Table 1. FIG. 2D shows the air flow performance curve of force velocity and differential pressure with a thickness of 0.0625″ and 0.25″ for the PE100 material shown in Table 1. FIG. 2E shows the air flow performance curve of force velocity and differential pressure with a thickness of 0.0625″ and 0.25″ for the PP50 material shown in Table 1. FIG. 2F shows the air flow performance curve of force velocity and differential pressure with a thickness of 0.0625″ and 0.25″ for the PP150 material shown in Table 1.

The construction details of the heating disc 100 shown in FIG. 1A and FIG. 1B are that the heating disc material be made of iron converting to ferrous oxide, with salts and inert materials resulting in the following reaction 4Fe(s)+3O₂(g)→2Fe₂O₃(s). The salts and inert materials act as reaction accelerants or retardants as needed to slow or speed the reaction and as dispersants to help create a uniform mixture within the polymer matrix. Once the reaction of 4Fe(s)+3O₂(g)→2Fe₂O₃(s) is driven to completion the individual reaction cannot easily be reversed, so each disk becomes a single use disposable product.

Still referring to FIGS. 1A & 1B, there is shown the heating disc 100 with a curvature. Although the disc appears to be solid in nature, there is porosity available to allow access of air to the surface and orifices of the heating material. The chosen porosity along with the ingredient choices and density of the disc when manufactured, and available surface area of the heating material, dictates heat conversion rates.

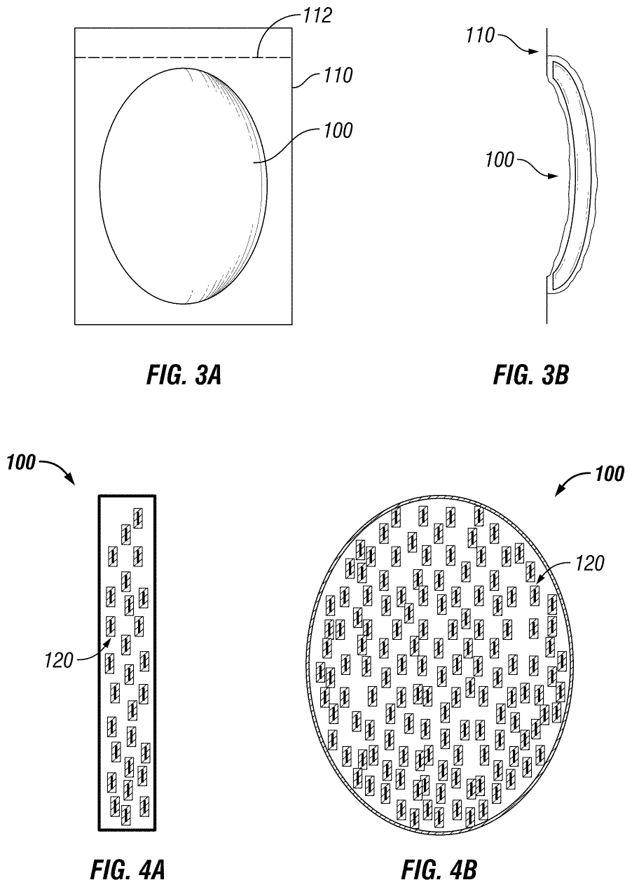

In more detail, still referring to the heating disc 100, the disc shown in FIGS. 3A & 3B includes an overwrap material or barrier layer 110 for shipping and product storage until used by the patient. The barrier layer 110 provides an extended shelf life of at least 3 years and is needed to keep H₂O and air away from the heating disc 100 to prevent premature reaction of the heating material. A pouch tear strip 112 is provided where a user may tear the barrier layer 110 for actual used of the heating disc 100. Water Vapor Transmission Rate (WVTR) measures the transmission of water vapor through a material and is determined using a modulated Infrared Sensor ASTM F1249 Test Procedure. WVTR is measured in either grams/100 in²/24 hours or grams/m²/24 hours (according to the standard ASTM-E398). Oxygen Transmission Rate (OTR) is the measurement of the amount of oxygen gas that passes through a material over a given period of time and is determined using a Coulometric Sensor ASTM D3985, ISO 15105 Test Procedure. OTR is measured in either cm³/m²/24 hours or cm³/100 in²/24 hours. The barrier layer preserves the desired properties of the disc for at least three years. Lower OTR and WVTR rates are preferred. An example of materials with good storage properties are WVTR rates of 0.3 g/100 in²/24 hours @ 37.8° F.=90% RH, for PP and 0.35 g/100 in²/24 hours @ 37.8° F.=90% RH, for high-density PE (HDPE). While very good for WVTR, these have less attractive OTR rates at 150 cm³/100 in²/24 hours @ 25° C., for PP and 110 cm³/100 in²/24 hours @ 25° C., for HDPE. Typical long-term barrier layers 110 also include a layer of aluminum foil. This is a good example of why multi-layer commercial films are used for the product overwraps which include preferred transmission rates for both WVTR and OTR characteristics. When combined, these characteristics achieve longer shelf lives for three-year protection of a product sensitive to water and oxygen.

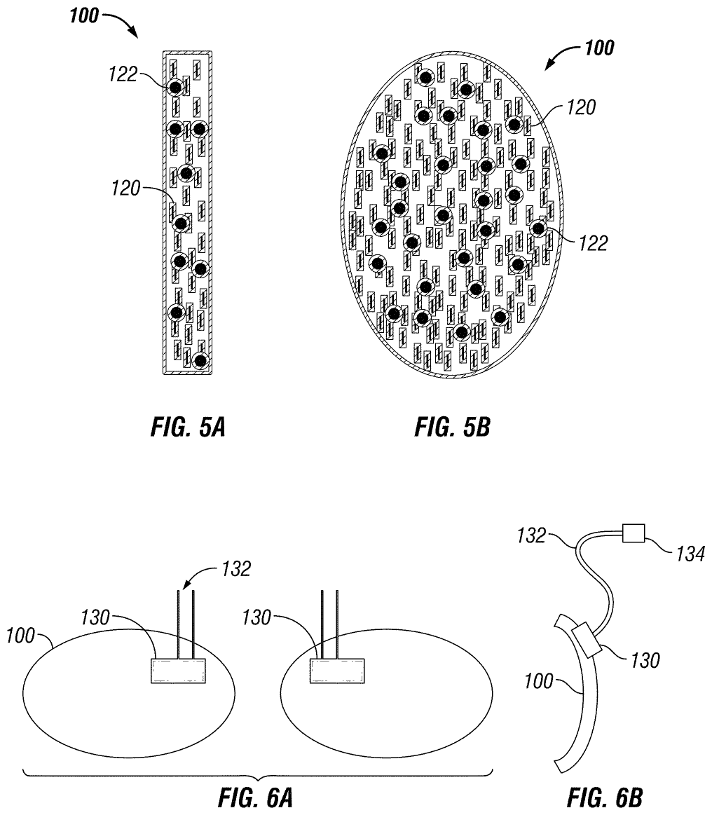

In further detail, referring to the heating disc 100 of FIGS. 4A-4B and FIGS. 5A-5B, the construction of the disc, meaning its shape, materials of construction, ingredient ratios of construction material, surface area, density and porosity make this disc a unique moldable heater product. Furthermore, the heating disk polymer support material, during manufacture, can be infused with nanoparticles of silica or metals to store and then release the heat generated by the FeOₓ heating material. This combination of components in the polymer matrix can be titrated and calibrated to transfer heat rate across the supporting materials and mask at a desired rate. Aluminum or other metal nanoparticles 120 are good heat conductors where the polymer is not. Silica or ceramic nanoparticles 122 act as a heat sink to store and slowly release the heat energy. Adding nanoparticles 120, 122 to the polymer, particularly the metals 120, also enhances desirable vibration transfer characteristics. The nanoparticles 120, 122 participate in structural integrity, also enabling a degree of structural pliability that allows for customized molding of the product to the individual patient's closed eyelid surface topography, as well as greater longevity of use for the product. This also translates into better product survival during transportation and during customer use. Polymer choices with good flexibility, shape memory, permeability and good heat transfer for desirable product characteristics can be determined from Table 1.

According to at least one embodiment, there is provided a heating disc 100 with harmonic resonance frequency stimulation vibration which is designed for use or reuse in a treating physician's office. Referring to FIGS. 6A-6B, the heating disc 100 includes resonance frequency stimulation vibration generators (RFSVG) 130 to transfer shear forces or vibrational energy to the surface of the eyes. Those RFSVGs 130 could be a single unit or one for each eye. The heating disc 100 is designed to hold the RFSVG 130 and fit comfortable over the patient's head and apply gentle pressure sufficient to transfer resonance frequency stimulation energy to the closed eyelids, either to the inner surface of the eyelid or on the entire external eyelid skin surface. To transfer the energy (heat and vibration) directly to the eyelid surface, a sanitary and disposable single use coupling device contacts the patient's skin. Energy and control is provided to the heating disc 100 via lead wires 132 and a micro USB 134 or other suitable connector from a mobile control module.

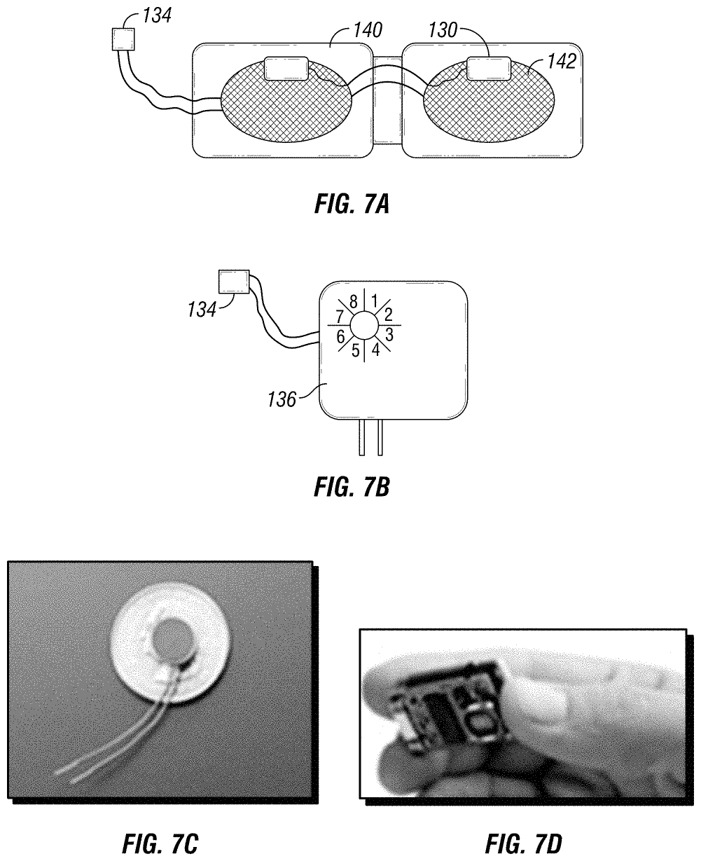

According to at least one embodiment, there is provided a heating mask 140, as shown in FIGS. 7A and 7B, with harmonic resonance frequency stimulation vibration which is designed for use or reuse in a treating physician's office. For purposes of illustration, a resusable heating mask 140 will be described in the following embodiments. Referring to FIG. 7A, the heating mask 140 contains a built-in heater element 142 or individual elements for each eye. The heater element 142 can include the heating disc 100. The system further includes RFSVGs 130 to transfer shear forces or vibrational energy to the surface of the eyelids. Those RFSVGs 130 could be a single unit or again one for each eye. The heating mask 140 is designed to hold these components and fit comfortably over the patient's head and apply gentle pressure sufficient to transfer resonance frequency stimulation energy to the closed eyelids, either to the inner surface of the eyelid or over the entire eyelid surface. To transfer the energy (heat and vibration) directly to the external eyelid skin surface, a sanitary and disposable single use coupling device contacts the patient's skin. Energy and control is provided to the heating mask 140 via a micro USB 134 or other suitable connector from a mobile control module. As shown in FIG. 7B, the heating mask 140 can include a vibration modulation controller 136 connected via USB 134.

The details of the heating mask 140 in FIG. 7A show a built-in resistance heater element 142 or metal wire woven into the mask designed with a heating element length to convert enough electrical input to the appropriate eyelid at optimum temperatures as detailed above. The resistance heater wire or metal wire can be woven into the fabric or structure of the heating mask 140 and has dual purposes: one is to generate heat when electrically energized and the other is to serve as a good vibration conductor. In some embodiments, the heater element 142 can include the heating disc 100. The resistant heater element 142 can be a single, moldable heater mesh having a shape similar to the heating mask 140.

According to at least one embodiment, power supply to the resistance heater can be accomplished for example through the USB connection 134 to a micro drive or mobile control module. Alternate power sources include disposable and rechargeable batteries. These batteries could be placed into the reusable mask if desired to eliminate cords extending from the reusable mask. A micro drive control board controlling the heater and resonator functions could be powered from a single supply voltage of 8-48 VDC, offering up to 100 W of peak power without any additional heat-sink. FIG. 7D is an example control board sold by Ingenia Motion Control. These boards combine the controller, drive, and stand-alone capability into a single unit with an incredibly small footprint. The control board preferably resides in a mobile system controller that is easy to carry around the physician's office. The mobile controller might combine the controller, drive, and stand-alone capability into a single unit with a small footprint. This is but one example of “off the shelf” components available to combine with the heating mask 140 and complete the frequency stimulation set-up and control of the heating mask 140.

According to at least one embodiment, the miniature RFSVG 130 induces a vibration through the coupling device to the surface of the eyelid. The control of vibration may include amplitude, a width, frequency, and where one or more of these parameters may be varied over the treatment period. The resonant vibration may have a frequency stimulation between about 2 Hz to about 270 Hz, between about 15 Hz to about 40 Hz, or between about 30 Hz to about 60 Hz. The resonant vibration may include a current having a pulse width or duty cycle between about 20% to about 80%. Vibration having the above-mentioned parameters may be used to treat one or more conditions, such as dry eye. Ideally in the physician's office, the controller would run through a range of pre-established frequencies and patterns. This range is to determine an individual patient's best response of resonance frequency stimulation to the applied vibration. This resonance frequency stimulation is the condition best suited to excite and mobilize an individual's flow of the Meibom lipids from the Meibomian glands. In some embodiments, anthropomorphic features and other characteristics of the patient, for example, eyelid laxity helps in the determination of the patient's personalized resonant vibration frequency. Non-limiting examples of anthropomorphic features and other patient characteristics include eyelid laxity, eyelid dimensions, eyelid mass, eyelid thickness, patient's race, patient's age, patient's sex, and any history of eyelid surgery. Non-limiting examples of anthropomorphic features and other patient characteristics also include MGD status, such as the percentage of clogged or plugged Meibomian glands, the degree of truncated Meibomian glands, and the quality of the Meibom lipid (i.e., thickness, turbidity, and clarity).

According to at least one embodiment, the tunable RFSVG 130 for the heating mask 140 may be provided by a number of different sources including sonic generators, electrodynamic or mechanical (such as cell phone vibrators) vibration generators. In some embodiments, the source is relatively quiet and able to deliver the vibrational energy through the disposable patient contacting coupling device to the underlying tissue. According to the concept of finding resonance to the patient's Meibomian glands and blocked oil glands, the frequency stimulation may be adjustable and tunable. There are a number of miniature vibration modules such as Adafruit, shown in FIG. 7C, which have the recommended range for patient individual tuning capabilities. This particular module has a 0-5 VDC range running at 11,000 rpm at the top end at 100 mA. The range may be reduced to 0, by reducing the voltage to the module, for example: 3 VDC @ 60 mA is linear and at 60% or 6,600 rpm. The power requirement is very low and may be operated by remote supplied energy or in-eye-mask supplied battery power.

According to at least one embodiment, direct heating of the eyelids and adjacent areas may be achieved by weaving a resistance NiChrome heater wire as the heater element 142 into the heating mask 140 as shown in FIG. 7A. The energy to heat the heating mask 140 to the desirable temperature may be provided by a USB connector 134. The resistance across the NiChrome heater wire dictates the heat converted by the amount of energy provided. The heating mask 140 has a compartment, or slip on top of the heater area, for the imbedded RFSVG 130. The heat control from the heater wire 142 can also be controlled by the same type device controlling the RFSVG 130.

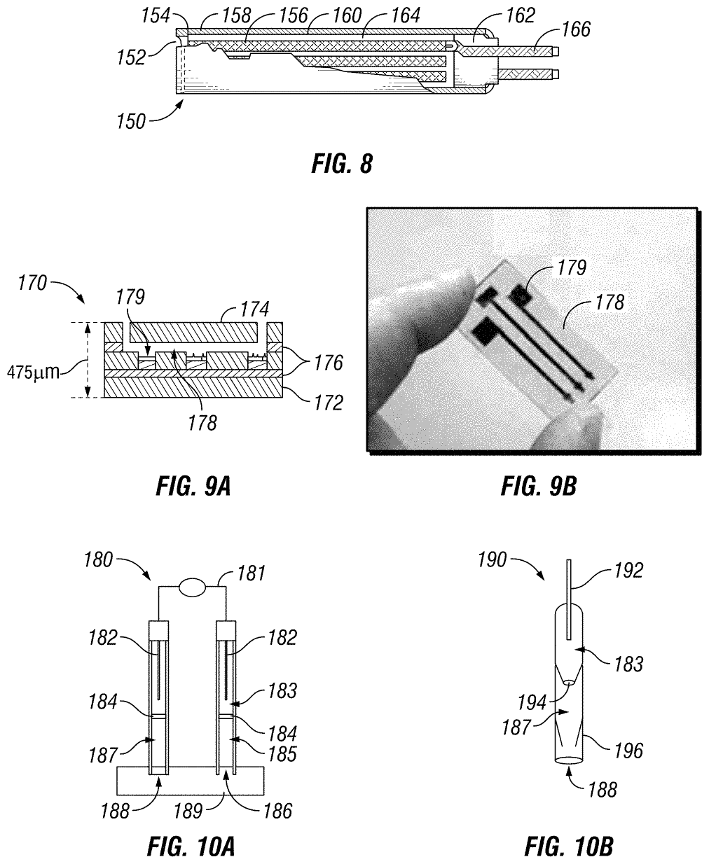

Referring to FIG. 8, the patient's Meibomian gland can also be warmed effectively and comfortably by use of a far infrared front end spot heater 150. Far infrared radiation may be directed to a precise location and the target area warmed to a precise temperature of about 40-46° C. Radiation is the most prevalent source of heat transfer in our universe and the Stefan-Boltzman law of radiation states that as the temperature of a heat source is increased, the radiant output increases to the fourth power of its temperature. Although not to be bound by any theory, this may suggest that far infrared targeted heating is a logical approach to Meibomian gland warming.

According to at least one embodiment, the far infrared front end spot heater 150 is constructed to radiate heat from the far infrared end seal 152 made of heat transmitting material (thin metal face or substitute). Heat is transferred to the far infrared end seal 152 by a conducting plug 154. This plug 154 is in contact with the end seal 152 and is a designed mass of conducting material for storing and releasing the heat converted by a heating element wire 156. The exterior or sides of the spot heater 150 are comprised of heat resistant insulation material 158 allowing a user to comfortably hold the spot heater 150 without risk of uncomfortable temperature exposure. A thermocouple (not shown) might also be employed with this device and integrated into the spot heater 150 properly. The interior of the spot heater 150 includes a conducting packing material 160 all the way to the tip or the plug 154 through a ceramic cap 162. The heating element wire 156 is supported in the spot heater 150 by ceramic element supports 164 that function in a stability capacity providing little movement and adding longevity to the spot heater device 150. The electrical leads 166 are fed through the ceramic cap 162 providing support for the electrical leads 166 and temperature barrier characteristics. The insulated electrical leads 166 are comprised of insulated electrical wire with lead lengths ending in a USB connector 134 for operating the spot heater 150 in the physician's office.

According to at least one embodiment, the heating mask 140 is comprised of soft, comfortable fabric like materials with an adjustable band to help the heating mask 140 reside in the appropriate location on the eyes. A moldable coupling device is a component for the heating mask 140 to provide a sanitary, possibly sterile, skin contacting surface for individual patient use. This single use, disposable coupling device will transfer the generated thermal and vibration energy generated by the heating mask 140 effectively to the eyelid surface. In some embodiments, the coupling device is composed of hydrogel, similar to a hydrogel dressing, possibly contained in a support structure or quilted construction to assure even distribution and intimate contact across the skin contacting regions. The hydrogel composition and water are controlled to best achieve this transfer, and add a controlled amount of moisture to the eyelids and lashes, with the added benefit of loosening debris on the eye lashes. According to at least one embodiment, the hydrogel layer makes direct skin contact. In alternate embodiments, the hydrogel could be constrained behind a thin moisture permeable barrier layer. In other embodiments, the coupling device is composed of a hydrogel sheet, and more particularly includes tea tree oil for treatment of, for example, demodex (i.e., mites) infestation of the eyelashes, which is common in blepharitis, Meibomian gland dysfunction, and dry eye disease.

Construction of the coupling device would allow hand molding to an individual's face, periorbita, and features or gentle reforming could be applied from pressure by the eye heating mask 140. The disposable coupling device would be easily replaceable in the heating mask 140 for use by a new patient. The coupling device would be prepared for long term storage using the barrier layer technologies described for the heating disk 100 and could be sterilized to a 10⁻³ or higher sterility assurance level (SAL).

As explained with the heating disk 100 above, this hydrogel layer could incorporate a mixture of particles to facilitate well dispersed heat transfer, heat sinking and bi-directional vibration energy transfer.

Alternatively, the coupling device could be made from thin layers of natural materials and fibers to create a comfortable and breathable surface against the skin. The heating mask 140 could be any number of fiber materials known to be breathable, such as cotton, linen, bamboo, or hemp. Other cloth fabrics from synthetic materials are also breathable and moisture transportable. Non-limiting examples include base layer clothing made from polyester and polypropylene. Filler materials inside the coupling device could be also made of breathable, natural fillers. The filler material may allow the heat to pass to the contact surface but also the vibration energy. Possible natural fillers, in small chunks or fibers, include bamboo fiber, small dried beans, quinoa, rice, and hemp. Size and size distribution of the filler material can be optimized to determine the best options for transmitting the vibration energy. Also possible are quilted fabric layers using various fillers to provide the loft in the quilt and non-woven felt materials.

According to another embodiment, the coupling device would apply moist heat to the surface. A source for the moist water vapor could be the hydrogel. As heat energy from the heating mask 140 transfers to the coupling device, water in the hydrogel or natural filler turns to vapor and crosses a moisture permeable barrier to the contact surface.

Alternately, reservoirs of water could be constructed into the coupling device to interact with the heat source.

According to at least one embodiment, a microfluidic enabled sensor shown in FIGS. 9A and 9B can be included at the patient's eye lid interface and be responsive in real time to track changes in Meibomian fluid flow. Changes in flow rate are induced by variation in vibration frequency stimulation from the RFSVG; the objective being to determine the best vibration parameters for an individual patient. The sensor provides analysis of very small samples and environments such as the Meibomian gland with the ability to measure very small change in flow. The chemical sensor is an ultra-sensitive yet simple sensor integrated into a microfluidic device, incorporating polymer-based Meibomian fluid selective liquid-contact and polymer-based solution-selective electrodes. The target component in the Meibomian fluid for the sensor analysis could be specific proteins, lipids, or other biomarkers produced with the flow of the fluid. In-situ sensors enable analysis of very small samples and environments such as the Meibomian gland at work with the ability to realize potentiometric output from very small changes of fluid flow utilizing liquid-contact electrodes, such as electrodes shown in FIGS. 10A and B. It is possible to incorporate the miniature RFSVG and the miniature integrated chemical sensor into the same device.

According to at least one embodiment, one form of the chemical sensor 170, shown in FIGS. 9A and B, has a number of layers formed by polymethylmethacrylate (PMMA) construction. This sensor has a lower PMMA layer 172, a top PMMA layer 174, a sensor liquid entrance, pressure sensitive adhesive (PSA) 176, conducting polymers, thermal pressure laminating, and CO₂ laser-generated 400 μm width fluid flow channels 178. The height of the microfluidic chemical sensor 170 is about 475 μm. A solution reactive material may be needed in the channels for detection on the Meibomian gland fluid selective catalyst layer 179. The deposition of solution reactive materials in the channeling is accomplished by electrode sputtering, if metallic. This process is well known in the art but has yet to focus on Meibomian gland issues as a target for patients until now. In this configuration, the data recorded relates to the presence of fluid.

Referring to FIG. 10A, a miniature solution selective electrode (SSE) 180 with integrated dual chemical sensors with potentiometric output is displayed. The chemical sensor uses a reference electrode 181 consisting of an inner reference half-cell 182, a reference solution 183, a diaphragm 184, bridge solution 185 with the diaphragm 184, a capillary or sleeve 186 at the entrance. The SSE 180 consists of the inner reference half-cell 182, the reference solution 183, the diaphragm 184, an inner filling solution 187, and a solution selective membrane 188. The sleeve 186 and the solution selective membrane 188 are in contact with a fluid sample 189. This miniature chemical sensor, when placed on the surface of the Meibomian gland, detects the duration of flow for patient diagnosis. Electric potential output from the SSE 180 is connected to a data acquisition module (not shown) capable to retrieve data up to 100 Hz speeds, real-time. This may allow real-time data mapping of the patient's Meibomian gland fluid, proteins, biomarkers, or lipids, which all are tracked to define each patient's individual characteristics and medical needs.

According to at least one embodiment, a single element version of the miniature integrated chemical sensor 190 with potentiometric detection, shown in FIG. 10B, is also applicable. It includes a solution sensing wire material 192, the reference solution 183, a separation plug 194, the inner filling solution 187, a SSE body 196, and the selective membrane 188. This single element miniature integrated chemical sensor 190 may be placed on the Meibomian gland and record data at the same rate as the SSE 180.

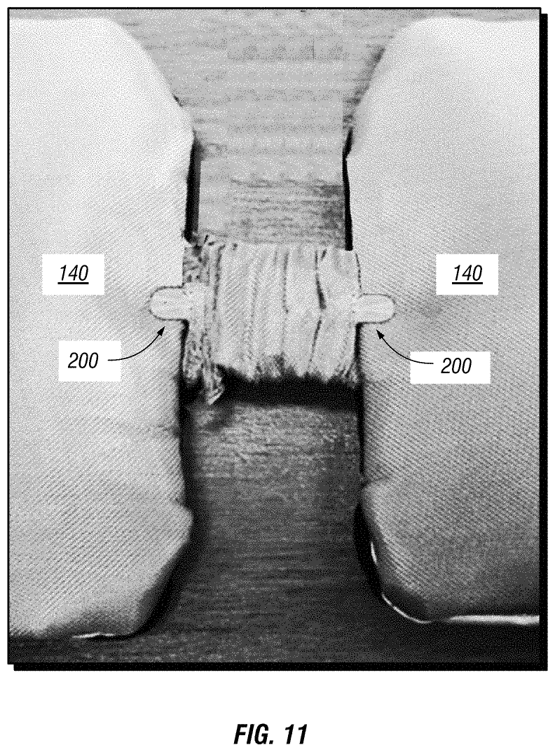

Referring now to the disclosed devices in more detail, in FIG. 11, there is shown the center connecting part of the mask, demonstrating a shape memory bridge 200 component allowing the patient flexibility forming the heating mask 140 to the patient's particular facial and periorbital features and surface topography. This allows better contact surface area by the mask's eye side surface to the eyelid thus achieving better heat transfer to the eyelid surface. According to at least one embodiment, the heating device 100 is manufactured to fit comfortably in the heating mask 140, conforming to the natural shape of the closed eyelid surface, and surrounding periorbital area. One suitable shape is an oval configuration relating to the shape of the ocular surface, other shapes, such as rectangle or square shapes that are larger than the oval shape, are also suitable.

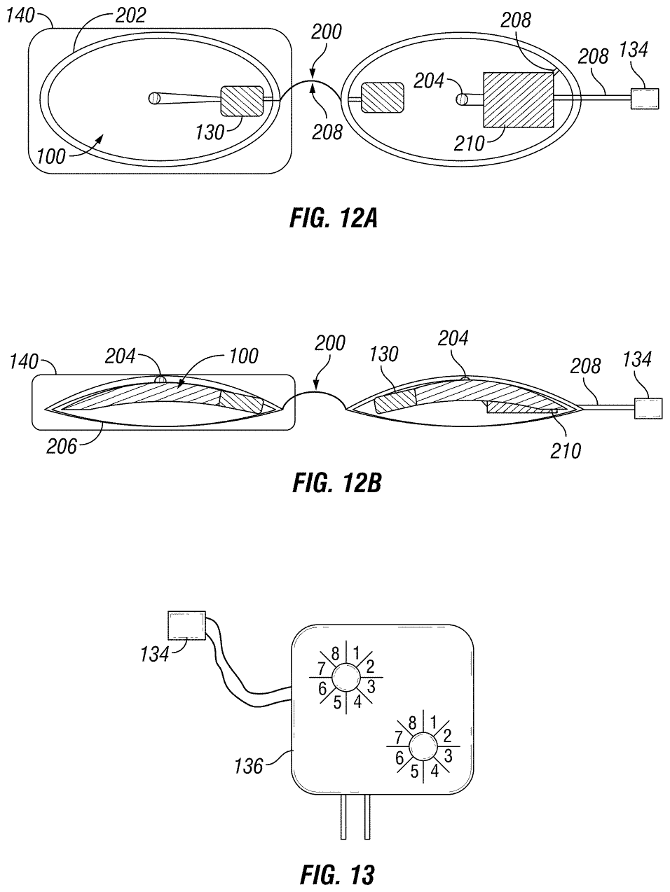

In further detail, referring to the devices disclosed in FIG. 12A and FIG. 12B, there is provided a front and top view of the moldable heating device 100 incorporated in the heating mask 140 with the RFSVG 130 located in one possible location in the moldable heater device 100. The drawing further details the layer of material against the skin 202 allowing better heat and vibration transfer via conductive heat transfer to the skin surface from the heating mask 140. Also shown is the shape memory bridge 200 allowing the patient flexibility forming the heating mask 140 to the patient's particular facial and periorbital features and surface topography. The heating mask 140 configuration shown in FIGS. 12A and 12B incorporates a single thermocouple 204 in each side of the heating mask 140 along with the RFSVG 130. In some embodiments, the mask material located on the outside layer of the heating mask 140 is made of heat reflective material 206 and is purposed to reflect and direct heat from the heating device 100 inward toward the eyelid surface. Reducing heat loss to the outside of the heating mask 140 has advantages such as better temperature control, patient comfort, and overall better temperature distribution over the eyelid surface area. The single thermocouples 204 are imbedded in the inner most layer against the eyelid and record the temperature in that location. The two dissimilar conductors produce a temperature dependent voltage carried via mask wiring 208 to the thermocouple multiplexing circuit board 210 where the signal is conditioned and carried via the USB connector 134 to a remote monitoring device or display for viewing or analysis. In further detail, referring to the devices of FIGS. 12A and 12B, the location of the RFSVG 130 is pre-positioned by the manufacturing process of the heating device 100. The pocket is sized for the RFSVG 130 and associated power sources and connections. The RFSVG 130 can be a reusable electrical device, which snaps into place for the duration of the one-time use or reusable heating device 100 and is then removed before the one-time use or reusable heating device 100 is removed.

FIG. 13 shows a rendition of further capabilities of the RFSVG 130 vibration modulation controller 136 incorporating a temperature controller for the electrical heater element 142, described in FIGS. 7A-7D. The vibration modulation controller 136 can be operated by the user or the physician. Strict control over the electrical heater element 142 allows flexibility over the range of temperatures used in dry eye therapy. The signal is carried out and power is provided via the USB connector 134.

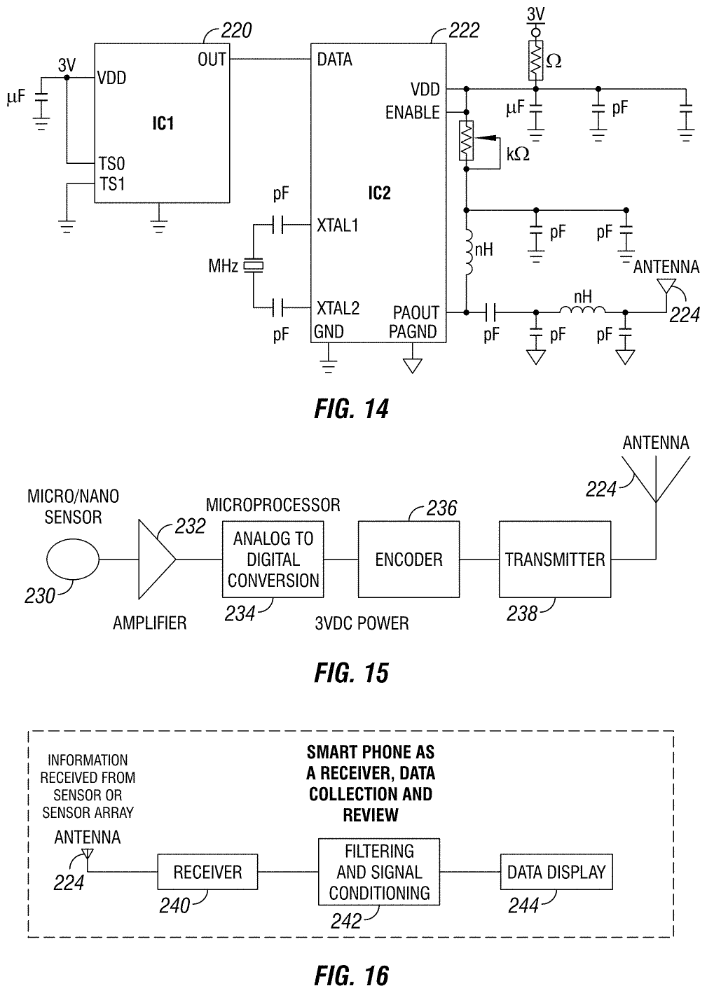

FIG. 14 shows an example electrical schematic showing the circuitry required to localize and transmit sensor information via antenna to a smartphone or smart device receiver, processor, and display. This technology has been well developed and is used to communicate collected data from sensors and sensor arrays via a smartphone or smart device app to a smartphone or smart device receiver, processor, and display. Sensor information collected by IC1 220 is transmitted to IC2 222. IC2 222 processes sensor information and further transmits the processed sensor information wirelessly via antenna 224.

FIG. 15 and FIG. 16 are block diagrams showing the path to transmitting and receiving sensor data to a smartphone or smart device receiver, processor, and display. Block diagrams are included as an assistance feature to better understand or conceptualize the embodiments disclosed herein.

According to at least one embodiment, referring to FIG. 15, analog data generated by the sensors or sensor array 230 is amplified via an amplifier 232 and converted into digital data streams via an analog-to-digital conversion (ADC) microprocessor 234. The digital data streams are encoded via an encoder 236 and then transmitted using a transmitter 238 wirelessly via an antenna 224.

According to at least one embodiment, referring to FIG. 16, wirelessly transmitted data, originated from the sensors or sensor array 230, is received using a receiver 240 via antenna 224 and is then filtered and conditioned for data display 244 using a signal filter and conditioner 242. In some embodiments, a smartphone or smart device is the receiving device. The smartphone or smart device is capable for filtering and conditioning the wirelessly transmitted data, and is also capable for displaying the processed data.

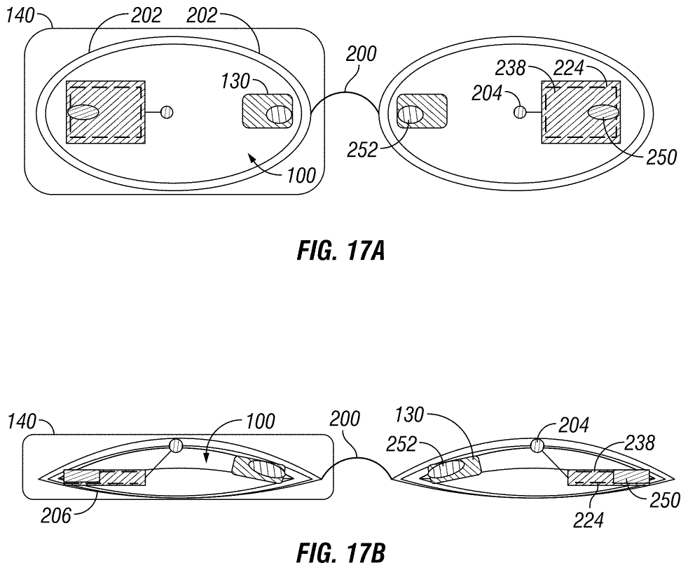

FIGS. 17A and B, still referring to the device in FIGS. 12A and B, show a single temperature sensor 204 location and RFSVG 130 in each ocular cavity along with localized transmission power source 250 and RFSVG power source 252 to energize the circuitry and frequency stimulation generator, respectively. In some embodiments, the wireless transmitter 238 is powered locally by a 3 VDC one-time-use or replaceable battery 250 where the thermocouple 204 data is transmitted via an antenna 224 to the patient's smartphone or smart device. The temperature sensor 204 is located in a forward proximity position to the eyelid for reliable and accurate temperature data collection. In some embodiments, the shape memory bridge 200 has no additional electronic connections in the wireless transmittal version. This configuration could also employ a remote power source via USB 134 where the shape memory bridge 200 would carry additional circuitry, corresponding to mask power and communication wiring 208, to transmit the temperature data.

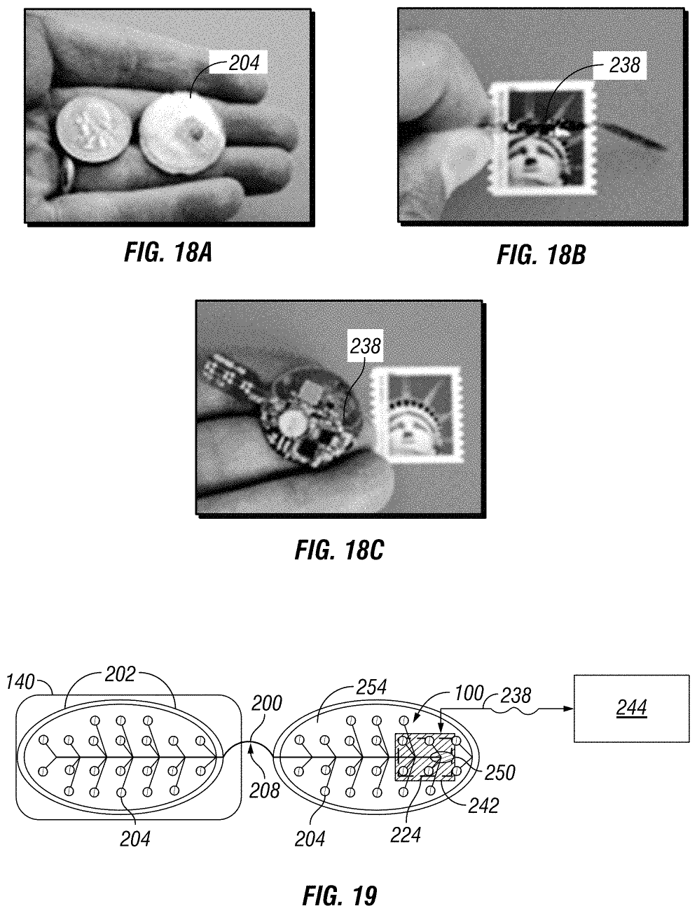

FIGS. 18A-18C show photographs of the microtechnology for collecting temperature data, for conditioning and transmitting the data to a smartphone or smart device. FIG. 18A shows the single thermocouple 204 next to a quarter for size comparison. FIG. 18B shows a side view of the data transmitter 238. FIG. 18C shows a front view of the data transmitter 238. Postage stamps are displayed for size comparison in FIGS. 18B-18C. The micro-components are easily integrated into the heating mask 140 for enhanced characteristics and utility of the proposed device.

FIG. 19, still referring to the device in FIGS. 12A-12B, shows a configuration with multiple thermocouple 204 positions, signal conditioning 242, and wireless transmission 238 of temperature data to a smartphone or smart device, wireless receiver 240, processor, and data display 244. Also provided is a flexible printed circuit board 254, which includes the thermocouples 204. In some embodiments, taking more temperature points in the given area provide a better temperature profile of the amount of energy and energy distribution produced by the heating device 100. The heating device manufacturer may focus on a more precise heater product and tailor the heater device 100 to the individual patient. In other embodiments, still referring to FIG. 19, a micro-frequency stimulation generator, such as the RFSVG 130, may be incorporated, which may be powered by a remote or localized power source, a replaceable battery, or USB connection 134.

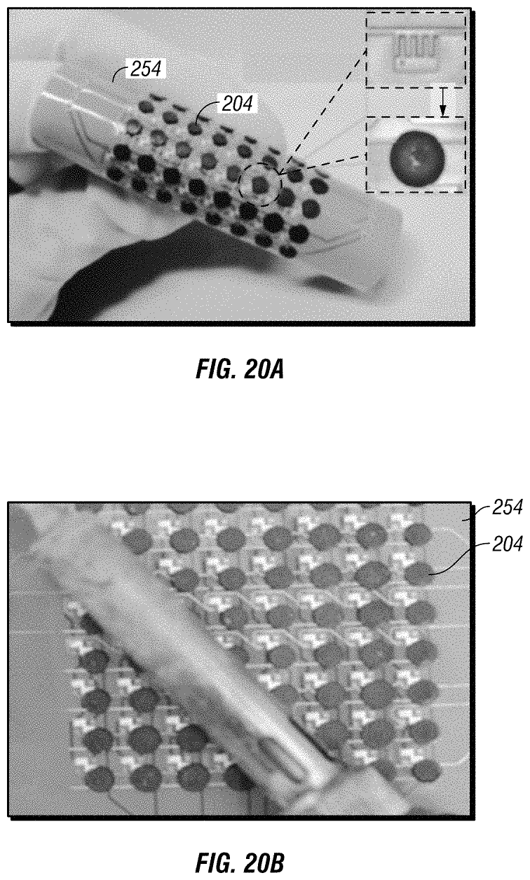

FIGS. 20A and 20B show a flexible printed circuit board 254 with closely aligned temperature sensors 204. This embodiment demonstrates how flexible the heating device 100 may be and how it may be used in the heating mask 140. In some embodiments, graphite-polydimethylsiloxane composite is dispensed on flexible polyimide films. A sensor array 204 may be, for example, an array with 64 sensing cells in a 4×4 cm² area. Interdigitated copper electrodes are patterned on the flexible polyimide substrate to determine the resistivity change of the composites subjected to ambient temperature variations. The flexible circuit operates with no signs of degradation in temperatures from about 30° C. to about 110° C.

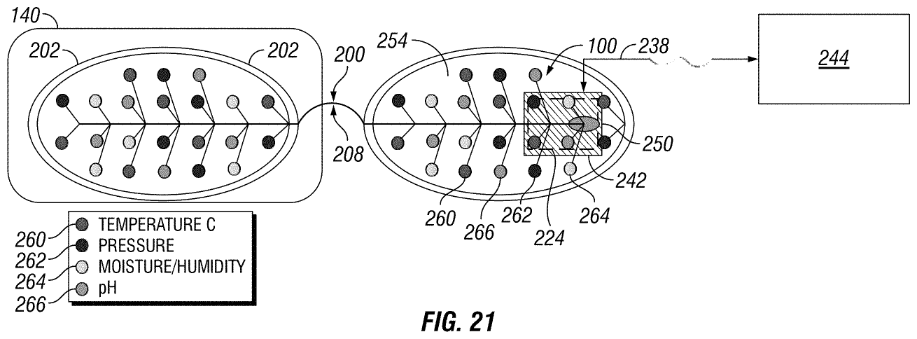

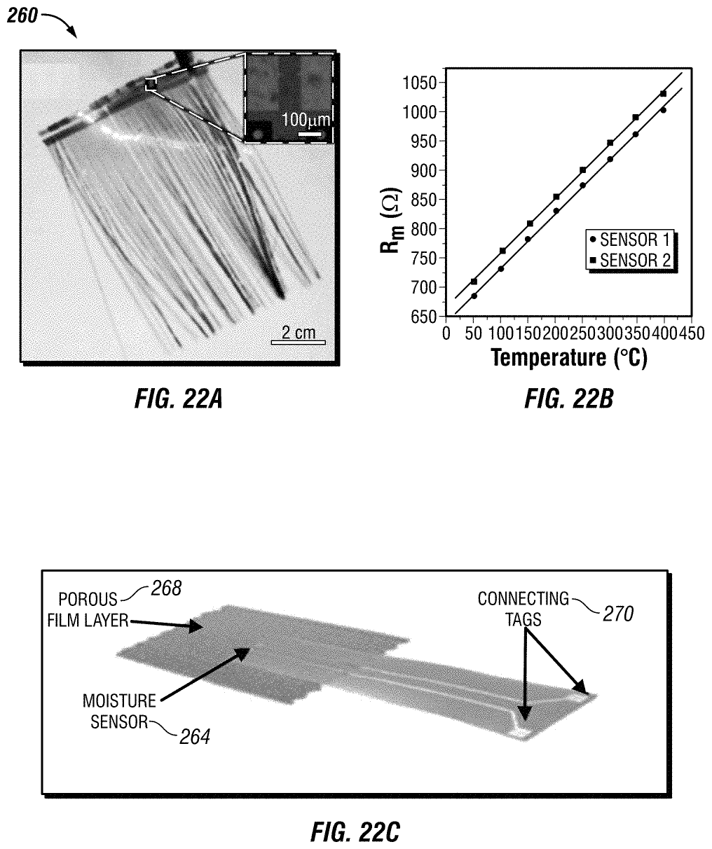

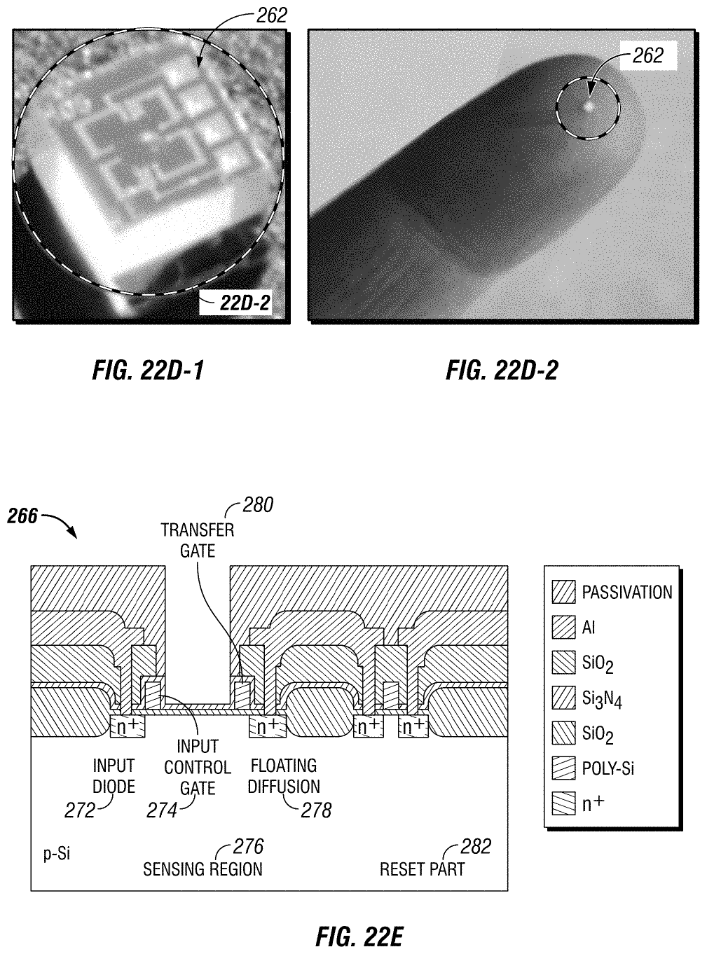

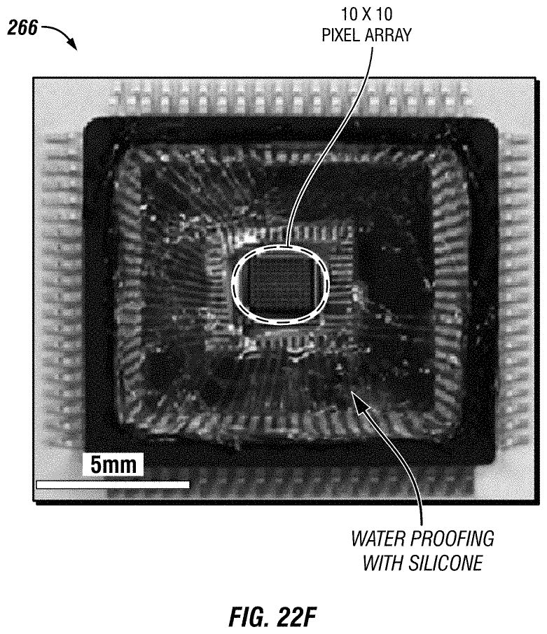

FIG. 21 still referring to the device in FIGS. 12A-12B, shows, in further detail of multiple sensor application, an embodiment where multiple types of sensors are imbedded on a single array 230 of a flexible printed circuit board 254. FIGS. 22A-F show embodiments of various types of sensors 230 that are capable of measuring, conditioning, and transmitting data for temperature 260, pressure 262, moisture/humidity 264, pH 266, and vibration frequency stimulation parameters. Application of these multiple types of sensors has been demonstrated for wound management. In some embodiments, data generated and collected from all of these sensors is conditioned and transmitted using wireless technology to a smartphone or smart device via an app. Sensors 230 are miniaturized for use in the ocular region via the heating mask 140.

Temperature sensors 260 used in embodiments of this disclosure is depicted in FIG. 22A. FIG. 22B shows a performance graph of the miniaturized temperature sensing device 260. As shown in FIG. 22B, electrical resistance (in ohms) of the temperature sensor 260 corresponds with the temperature. In some embodiments, temperature sensors 260 include a printed miniature format showing connection leads for data acquisition beyond the printed circuit board 254.

Referring now to FIG. 22C, an embodiment of a moisture/humidity sensing device 264 used in the mask 140 is shown. Moisture is an important parameter considering dry eye syndrome, sensing a patient's moisture level would be helpful for the physician to address the need whether to add additional moisture to the treatment. Moisture plays an important role in determining improvement for dry eye syndrome. In some embodiments, an additional layer of an aqueous solution provided and measured by micro-sensors 264 would benefit the treatment as adequate fluid balance is achieved. A continuous moisture level measurement provides informed decisions about treatment. In some embodiments, moisture is measured by a pair of replaceable, one-time use silver chloride electrodes. After a period of about 10 to about 30 seconds, the sensor 264 measures the moisture level by recording the impedance value shown in the onboard electronics. The porous film layer 268 surrounding the sensor is bi-functional where the porous film layer 268 allows moisture to reach the sensor 264 and provides mechanical support for the sensor 264 in-situ. Data generated is then transmitted via connecting tags 270 to a smartphone or smart device for processing and display. In some embodiments, a replaceable sensor is used when recommended as part of the sensor array 230.

Referring now to FIG. 22D-1, a pressure sensor 262 is shown including a miniaturized diaphragm-based silicon pressure sensor. A Wheatstone bridge unit is incorporated using silicon-on-insulator technology. Pressure applied to the surface of the closed eyelids is a parameter for determining patient comfort and mask tightness when treating dry eye syndrome. There are a number of miniaturized pressure devices 262 being manufactured in industry and size is of concern when putting the multiple sensor array 230 into use. FIG. 22D-2 shows a photograph of a miniature pressure sensing device 262 that is powered by a small local source and provides data to a smartphone or smart device via wireless technology. On the right hand side of FIG. 22D, the miniature pressure sensing device 262 is placed on a fingertip for size comparison.

FIG. 22E shows a depiction of a miniaturized pH sensing device 266. FIG. 22F shows a photograph of the miniaturized pH sensing device 266, where the areas other than the 10×10 pixel array are waterproofed with silicone. A wide range of pH measurement approaches are available, such as pH-sensitive polymers, ion sensitive field-effect transistors, near infrared spectroscopy. In some embodiments, a two-dimensional pH image sensor is manufactured by employing a CMOS fabrication process. Referring to FIG. 22E, the pH sensing device 266 includes an input diode 272, input control gate 274, ion sensing region 276, floating diffusion region 278, transfer gate 280, reset switch 282 and a source follower circuit. Employing CMOS technology, as shown in FIG. 22E, layers of poly-Si, SiO₂, Si₃N₄, Al, and a passivation layer can be deposited and patterned on a base substrate. In some embodiments, an RFID-based wireless optical chemical pH sensor 266 with contactless power and data interface may be employed. The optical chemical pH sensor 266 is based on the differential absorbance of light in bromocresol green (BCG) in which BCG is pH sensitive.

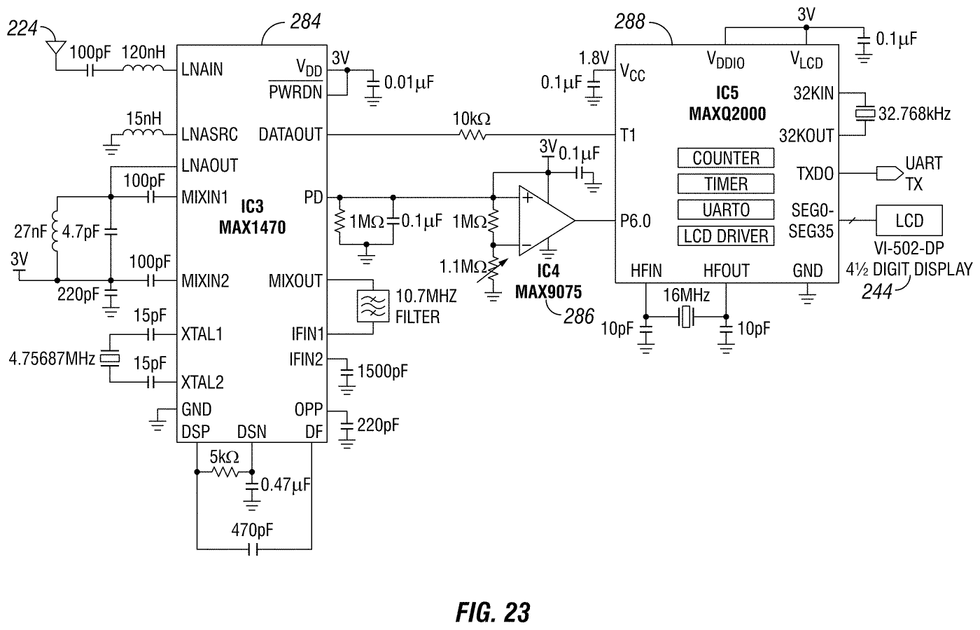

Referring to FIG. 23, an exemplary commercial wire schematic is shown for a smartphone or smart device to receive, process and display information from a wireless sensor array. Digitally-converted and wirelessly transmitted sensor data is received by IC3 284 via antenna 224. The data is further processed by IC3 284, IC4 286, and IC5 288. IC5 288 transmits the processed sensor data to the display 244 for visual display. Those skilled in the art would recognize that various means exist for a smartphone or smart device to receive, process and display information from a wireless sensor array.

According to at least one embodiment, vibration and temperature are controlled by pulse width modulation (PWM). Switching-voltage regulators employ PWM control for the switching elements. The PWM signal is either generated from a control voltage (derived from subtracting the output voltage from a reference voltage) combined with a saw tooth waveform running at the clock frequency for the voltage-mode regulator, or by adding a second loop feeding back an inductor current for current-mode control. Devices employ techniques such as voltage feed-forward for voltage-control designs and slope compensation for current-mode units.

In some embodiments, both types of topology are employed in the system. In other embodiments, component parts are linked together in the system. Voltage-mode control switching regulators are used in some embodiments when wide-input line or output-load variations are desired, under light loads (when a current-mode control-ramp slope would be too shallow for stable PWM operation), in noisy applications (when noise from the power stage would find its way into the current-mode control feedback loop), and when multiple-output voltages are needed with good cross regulation.

In some embodiments, current-mode control devices are used for applications where the supply output is high current or very-high voltage; the fastest dynamic response is sought at a particular frequency, input-voltage variations are constrained, and in applications where cost and number of components must be minimized as in the innovations stated here within.

According to at least one embodiment, the reusable mask 140 and even the entire system is suitable for mobile control, in which the device is easily hand held and carried for patient use. Control may also be driven by a smartphone or smart device using operating systems such as iOS, Android or Windows mobile, or other similar interfaces. Mobile medical interfaces are used in products such as a Zebra MC40 Mobile Computer. Similar platforms, or other Wi-Fi, cell phone or Bluetooth connected interfaces can be used to control the patient's first in-office use of the system. In some embodiments, a range of frequencies are tested and output data from the sensors is stored. The data storage and its associated algorithm may determine the best treatment mode for following office visits or transfer an optimal program to an at-home unit. These mobile interfaces further create efficiencies for the office staff by automatically storing patient records to the electronic medical records (EMR) of the first and subsequent uses. These records include patient name, time and date of use, frequencies explored and sensors output during that time. The at-home unit would also serve as a record of patient compliance to prescribed therapy.

According to at least one embodiment, the harmonic resonance heating mask 140 is preferentially supplied as a kit. Kits include one or more devices, and varying numbers of replacement heaters depending on kit size. Kits may include both elements of the one-time use components and reusable components. In some embodiments, for example, a kit might include the one-time use heating element 100, the reusable miniature harmonic resonance frequency stimulation generator 130 pairs that fit into the eye patch component and plug into a USB 134 port and the one-time use coupling device. Kits may be provided to a patient during an office visit as the equipment used to define the correct resonance frequency stimulation would be available in the practitioner's office. Commercial kits may also be provided with very specific frequencies and then purchased directly by an informed customer.

According to at least one embodiment, as mentioned in describing the mobile controller, after a patient's first use of the system in the physician's office, the patient may be prescribed to continue therapy on a more frequent basis at home. As an alternate embodiment, this system could be simplified for the home user. This system would have a reusable mask 140 with single use disposable, or reusable built-in heating elements 100 and resonance frequency stimulation generators 130, accommodate an optional disposable coupling device and come with appropriate power supply and control, including a mobile and wirelessly connected controller. The at-home monitoring system would not require a full range of vibration frequencies as the optimal frequency stimulation and pattern was determined in the original office use and that pattern is programmed into the individual user's system. Similarly, the full sensing capability is not needed for home use. A cell phone, Wi-Fi or Bluetooth connected controller may also create a record of use for the patient's EMR. In some embodiments, patterns of noncompliance or misuse may create an alert to go directly to the patient and/or back to the treating physician.

A further alternate embodiment may include a system that employs single use heating discs 100. This could be used for either the office based or home use products. The disposable heating disc 100, being hand moldable to conform to an individual's anatomy, would fit into the pocket in the heating mask 140. This heating disc 100 element could also be built into and supplied as part of the coupling device that contacts the skin and comprises a combined single disposable item. As shown in FIGS. 6A and B, in some embodiments, a moldable heating disc 100 with a low cost RFSVG 130 may be located in the disposable heating disc 100 and connected via the USB 134 port for control and energy. A reusable RFSVG 130 (mini-vibrator) can be placed in the manufactured pocket location located in the moldable heating disc 100. This approach may be used for both the moldable gel and polymer eye piece.

According to at least one embodiment, there is provided a method of treating dry eye disease or MGD. These methods include the initial physician's office based use where optimal treatment parameters are determined and then stored for later use either in subsequent office visits or home use.

The advantages of the devices disclosed include, without limitation, that it is portable, easy to transport, reliably functions as intended, and is simple and convenient to activate and use. Another advantage is that it is easy to integrate these devices into a reusable face mask or eye patch because they are relatively small and lightweight, showing the parallel utility of the device components stated herein.

A further alternate embodiment may include an integrated real-time imaging device to detect optimal tuning of the RFSVG 130 to the particular patient eyelid and Meibomian glands. In some embodiments, for example, optoacoustic imaging or photoacoustic imaging is insensitive to photon scattering within biological tissue and, unlike conventional optical imaging methods, makes high-resolution optical visualization deep within tissue possible. A key empowering feature is the development of video-rate multispectral imaging in two and three dimensions, which offers fast spectral differentiation of distinct photo-absorbing moieties. In some embodiments, the imaging device provides a real-time-image-based assessment of the optimal settings for the miniature RFSVG 130 at which there is maximal movement of the eyelids, Meibomian glands, and lipid fluid within the Meibomian glands.

According to at least one embodiment, there is provided means for providing a physician and a patient with a metric related to the state of the dry eye disease being treated. This metric will correlate to the severity of disease, and may be measured and provided both before and after treatment. Increased sensitivity to light is a well-known proxy for severity of dry eye disease. According to at least one embodiment, there is provided a light sensor configured to measure light sensitivity of the eye being treated and to provide a subjective light sensitivity score as a diagnostic indicator.

According to at least one embodiment, there is provided a method in which prior to initiating a treatment, the patient looks at a target in the mask 140 or at a distance. A light-emitting diode (LED) with a controllable spectrum is mounted to a head-mounted mask. The LED in the mask will turn on at an adjustable initial setting. The patient adjusts the intensity to the maximum comfortable level, with a physical rheostat or other controller. Right and left eyes may be tested sequentially, or both eyes may be tested simultaneously. The light intensity setting is recorded electronically. At the end of the treatment, the patient is exposed to light and the light sensitivity measurement is performed again. Each time the patient uses the device, their pre-treatment and post-treatment light sensitivity is recorded electronically, and comparison made with the previous light sensitivity scores. The comparison provides an indicator of treatment success, as well as dry eye disease stability, improvement, or worsening. According to at least one embodiment, the system can include a feature to automatically increase or decrease the treatment duration and/or intensity based on the light sensitivity measure, and relative change from the previous light sensitivity value.

According to at least one embodiment, the heating mask 140 can be configured to fit a single eye. The single-eyed heating mask 140 can be configured to fit either the patient's right or left eye. In some embodiments, the heating mask 140 can include two single-eyed heating masks, one configured to the right eye and the other configured to the left eye.

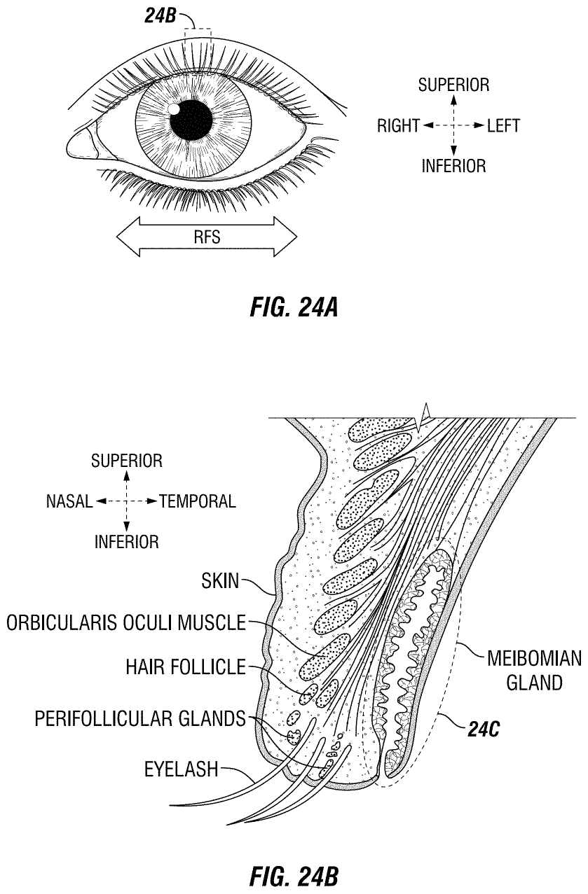

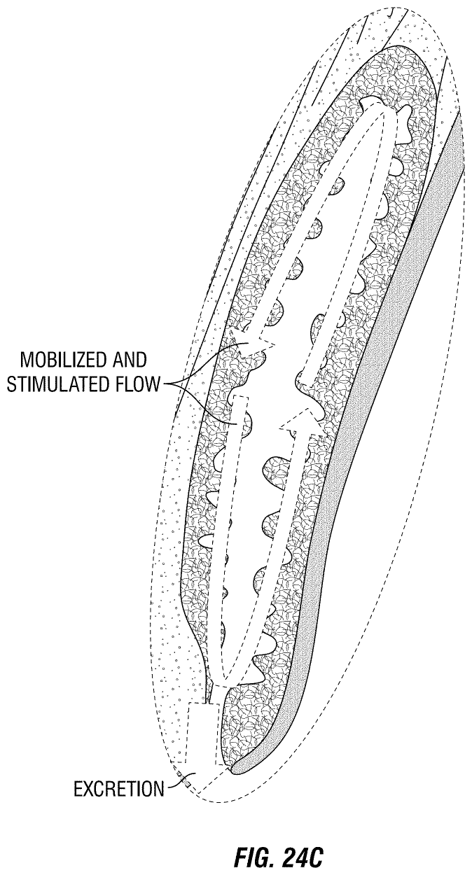

FIG. 24A shows a front view of a human eye. FIG. 24B shows an enlarged cross-sectional view of an upper eyelid of the human eye. FIG. 24C shows a cross-sectional view through a Meibomian gland of an upper eyelid of a human eye where the harmonic resonance frequency stimulation vibration mobilizes and stimulates flow of the Meibom lipids within a Meibomian gland. The mobilized and stimulated flow leads to enhanced excretion of the Meibom lipids.

According to at least one embodiment, the harmonic resonance frequency stimulation is a vector force. The harmonic resonance frequency stimulation exhibits traits substantially similar to a longitudinal wave. In some embodiments, the harmonic resonance frequency stimulation exhibits traits substantially similar to a longitudinal standing wave.

According to at least one embodiment, the primary axis of vibration is substantially parallel to the medial-lateral axis, as shown in FIG. 24A. The primary axis is tangential to the depth of the Meibomian gland duct, which is shown as the superior-inferior axis in FIGS. 24A and 24B. As shown in FIG. 24B, the primary axis of vibration is substantially tangential to the opening of the Meibomian gland duct. In some embodiments, the amplitude of the vibration, corresponding to the vector force, is configured to be substantially parallel to the medial-lateral axis, as shown in FIG. 24A. Accordingly, a RFSVG 130 may generate harmonic resonance frequency stimulation vibrating in the medial-lateral direction, which is substantially tangential to the depth of the Meibomian gland duct.

According to at least one embodiment, the primary axis of vibration can be substantially parallel to the superior-inferior axis, as shown in FIGS. 24A and 24B. The amplitude of the vibration, corresponding to the vector force, is configured to be substantially parallel to the superior-inferior axis, as shown in FIGS. 24A and 24B. Accordingly, a RFSVG 130 may generate harmonic resonance frequency stimulation vibrating in the superior-inferior direction, which is substantially parallel to the depth of the Meibomian gland duct.

According to at least one embodiment, the harmonic resonance frequency stimulation can be a superposition of two or more longitudinal vibrational waves. In some embodiments, the harmonic resonance frequency stimulation is a superposition of two longitudinal vibrational waves, where the primary axis of the first vibration can be substantially parallel to the superior-inferior axis while the primary axis of the second vibration can be substantially parallel to the medial-lateral axis, both axes as shown in FIGS. 24A and 24B. The amplitude of the first vibration, corresponding to the first vector force, is configured to be substantially parallel to the superior-inferior axis, as shown in FIGS. 24A and 24B. The amplitude of the second vibration, corresponding to the second vector force, is configured to be substantially parallel to the medial-lateral axis, as shown in FIG. 24A. Accordingly, a RFSVG 130 may generate two simultaneous harmonic resonance frequency stimulations, one vibrating in the superior-inferior direction, which is substantially parallel to the depth of the Meibomian gland duct, and the other vibrating in the medial-lateral direction, which is substantially tangential to the depth of the Meibomian gland duct.

According to at least one embodiment, the direction of the harmonic resonance frequency stimulation vector force can be selected by the device operator based on the degree of MGD (i.e., truncated gland ducts, clogged or plugged gland orifices versus open gland orifices).

According to at least one embodiment, the harmonic resonance frequency stimulation mobilizes the Meibom lipids within the Meibomian glands. The mobilization is achieved by inducing shear forces using vibration at the resonance frequency or frequencies of the patient's eyelid and Meibomian gland complex. As shown in FIG. 24C, a flow of the mobilized Meibom lipids is generated as a result of the vibration. After the Meibom lipids are mobilized, natural eye blinking will lead to the excretion of these lipids.

According to at least one embodiment, the reusable mask can include a bladder-type coupling device to provide substantially full contact to the patient's individual eyelid and periorbital three-dimensional anatomy and surface topography. The bladder-type coupling device can be filled with gaseous or fluidic medium, or foam. Hydraulics or pneumatics can be applied to control the coupling device. In some embodiments, a hydraulic medium is used in the bladder-type coupling device, where the hydraulic medium has a viscosity suitable for conforming to the patient's individual eyelid and periorbital three-dimensional anatomy and surface topography. The hydraulic medium is suitable for transmitting harmonic resonance frequency stimulation vibration generated by the RFSVG 130 to the patient's Meibomian glands. The hydraulic medium is suitable for transmitting the vibration in any direction. In some embodiments, the bladder-type coupling device includes channels to provide direction control of the vibration. In some embodiments, temperature and pressure control of the reusable mask can achieved by hydraulically inserting the hydraulic medium into the bladder-type coupling device.

According to at least one embodiment, the heating mask 140 is operable to change configuration of applying heat and the resonance frequency stimulation vibration. In some embodiments, the heating mask 140 is operated such that heat (for example, provided by the heating disc 100) and the resonance frequency stimulation vibration (for example, provided by the RFSVG 130) are applied to the patient's eyelid area sequentially and alternately, but not simultaneously. The heating mask 140 can be internally or externally programmed to achieve this sequence. In some embodiments, the medical practitioner cannot override the programmed sequence. In other embodiments, explicit instructions, such as an instruction manual, can be given to the medical practitioner to operate the heating mask 140 by applying heat and the resonance frequency stimulation vibration sequentially and alternately.

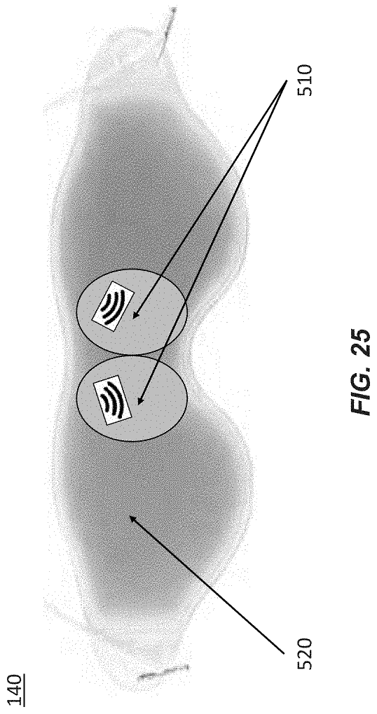

FIG. 25 shows an embodiment of the heating mask 140 including a neurostimulation unit 510. The heating mask 140 includes the moldable coupling device 520. In some embodiments, the moldable coupling device 520 includes hydrogel to assure even distribution and intimate contact across the skin contacting regions. The heating mask 140 conforms to the three dimensional topography of the eyelid surface. The heating mask 140 serves as a heat reservoir to deliver heat to the Meibomian glands through the eyelids. In some embodiments, the heating mask 140 includes the RFSVG 130 (not shown) to transfer shear forces or vibrational energy to the surface of the eyelids. Resonant frequency or non-resonant frequency stimulation vibration is delivered over the eyelids in a uniform manner to cause liquefaction and mobilization of the Meibum within the Meibomian glands.

According to at least one embodiment, the neurostimulation unit 510 includes the RFSVG 130 to transfer shear forces or vibrational energy to the nasal bridge area located between the eyes (as opposed to the cartilage area of the nose). Resonant frequency or non-resonant frequency stimulation vibration is delivered over the nasal bridge area through the nasal bone and to the sensory nerves (such as the anterior ethmoidal nerve in the nasal septum) to induce neuromodulation of the tear production reflex in the lacrimal functional unit (LFU), which encompasses the epithelium of the cornea and conjunctiva, the main and accessory (Wolfring and Krause) lacrimal glands, Meibomian glands, conjunctival goblet cells, and its corresponding innervation. Without being bound by any theory, stimulation in the nasal sensory nerves serves as an alternate afferent pathway for tear production reflex stimulation in addition to stimulation in the sensory nerves on the ocular surface. Stimulation is received in the sensory receptors located at the nasal mucosal epithelium, and travels to the superior salivatory nucleus through the anterior ethmoidal nerves, which is a branch of the ophthalmic division of the trigeminal nerve. The stimulation continues to travel through the pre-ganglionic fibers along the nervus intermedius to the pterygopalatine ganglion, and innervate the lacrimal glands, goblet cells, and the Meibomian glands. Such stimulation in the lacrimal gland results in an increase of the aqueous component of the tear, which is the middle layer component of the tear film. Such stimulation in the goblet cells on the conjunctiva results in the production of mucin, which is the innermost component of the tear film. Such stimulation in the Meibomian gland results in the expression of the meibum, which forms the external layer of the tear film and stabilizes thereof while preventing tear film evaporation.

Certain parameters of the resonant frequency stimulation vibration can be adjusted for optimal neurostimulation. For example, frequency can be adjusted such that the resonant frequency or non-resonant frequency stimulation vibration is optimized for bone conduction around the nasal bridge. Amplitude can be adjusted for optimal comfort and effectiveness. The primary axis of vibration can be substantially parallel to the medial-lateral axis, as shown in FIG. 24A, which is perpendicular to the bony surface of the nasal bridge, and may vary across the nasal bridge in order to maintain a perpendicular orientation to the nasal bridge.

Certain parameters of the heating mask 140 can be adjusted for optimal neurostimulation. For example, thickness can be adjusted to optimize the coupling between the external vibratory stimulus and the bony nasal bridge. Vibratory conductance of the material around the nasal bridge can be adjusted to optimize the transmission of external vibration resulting in bone conduction.

In alternate embodiments, the neurostimulation unit 510 includes an ultrasonic transducer (not shown) to transfer ultrasound directly to the sensory nerves (such as the anterior ethmoidal nerve in the nasal septum) to induce tear production reflex in the LFU. The ultrasonic transducer can be any ultrasonic transducer known in the art that is capable of neurostimulation and has a size suitable to be included as a component of the heating mask 140. The ultrasonic transducer can include an array of ultrasonic transducers that enable dispersed or focused ultrasound energy to maximally control the induced neuromodulation of the tear production reflex.

In alternate embodiments, the sequence and timing of the heating and vibration stimulation can be adjusted to obtain optimal tear film therapy. Specifically, the eyelid heating that reduces meibum viscosity may be applied first, while the resonant frequency stimulation vibration next mobilizes the meibum within the Meibomian Glands, followed by neurostimulation that, in addition to increasing mucin and aqueous production, induces expression of the mobilized meibum from the Meibomian Glands. This sequence, and length of time of each element of the sequence, as well as repetitions of all elements of the sequence, and total time of therapy, can all be adjusted to obtain an optimal therapeutic result.

While preferred embodiments have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed in practicing the invention. It is intended that any claims presented define the scope of the various embodiments and that methods and structures within the scope of these claims and their equivalents be covered thereby.

Embodiments described herein, therefore, are well adapted to carry out the objects and attain the ends and advantages mentioned, as well as others inherent therein. While a presently preferred embodiment has been given for purposes of disclosure, numerous changes exist in the details of procedures for accomplishing the desired results. These and other similar modifications will readily suggest themselves to those skilled in the art, and are intended to be encompassed within the spirit of the various embodiments disclosed herein and the scope of the appended claims.

Although the various embodiments have been described in detail, it should be understood that various changes, substitutions, and alterations can be made hereupon without departing from the principle and scope of the various embodiments. Accordingly, the scope of the various embodiments should be determined by the following claims and their appropriate legal equivalents.

The singular forms “a,” “an,” and “the” include plural referents, unless the context clearly dictates otherwise.

Optional or optionally means that the subsequently described event or circumstances can or may not occur. The description includes instances where the event or circumstance occurs and instances where it does not occur.

Ranges can be expressed herein as from about one particular value, and/or to about another particular value. When such a range is expressed, it is to be understood that another embodiment is from the one particular value and/or to the other particular value, along with all combinations within said range.

As used herein and in the appended claims, the words “comprise,” “has,” and “include” and all grammatical variations thereof are each intended to have an open, non-limiting meaning that does not exclude additional elements or steps.

As used herein, terms such as “first” and “second” are arbitrarily assigned and are merely intended to differentiate between two or more components of an apparatus. It is to be understood that the words “first” and “second” serve no other purpose and are not part of the name or description of the component, nor do they necessarily define a relative location or position of the component. Furthermore, it is to be understood that the mere use of the term “first” and “second” does not require that there be any “third” component, although that possibility is contemplated under the scope of the various embodiments.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The embodiments can suitably comprise, consist or consist essentially of the elements disclosed and can be practiced in the absence of an element not disclosed.

What is claimed is:

1. A moldable warming device, comprising:
a heating disc;
a first harmonic resonance frequency stimulation vibration generator (RFSVG);
a coupling device;
a mask configured to hold the heating disc, the first harmonic RFSVG, and the coupling device for use in parallel utility; and
a sensor array, the sensor array configured to determine tuning parameters of a vibration and heating profile of a user's individual eyelid, periorbital, and nasal three-dimensional anatomy and surface topography,
wherein the moldable warming device is configured to provide an eyelid surface and periorbital structures with therapeutic warmth,
wherein the first harmonic RFSVG is configured to provide tuned harmonic resonance or non-resonance frequency stimulation vibration over the user's nasal bridge area through the user's nasal bone such that nasal sensory nerves are stimulated to induce tear production reflex in the user's lacrimal function unit.

2. The moldable warming device of claim 1, further comprising:
a second harmonic RFSVG,
wherein the mask is configured to hold the second harmonic RFSVG,
wherein the second harmonic RFSVG is configured to provide the eyelid surface and periorbital structures with tuned harmonic resonance or non-resonance frequency stimulation vibration.

3. The moldable warming device of claim 1, wherein the coupling device is configured to contact the user's eyelid skin for heat transfer.

4. The moldable warming device of claim 1, wherein the coupling device comprises hydrogel.

5. The moldable warming device of claim 1, wherein the mask comprises a heat reflective material configured to reflect and direct heat from the heating disc toward the user's eyelid surface.

6. The moldable warming device of claim 1, wherein the sensor array comprises a thermocouple configured to record temperature of the user's eyelid surface.

7. The moldable warming device of claim 6, wherein the sensor array includes a flexible printed circuit board where the thermocouple is embedded therein.

8. The moldable warming device of claim 7, wherein the flexible printed circuit board comprises a polyimide film.

9. The moldable warming device of claim 1, wherein the sensor array comprises sensors selected from the group consisting of: temperature sensors, pressure sensors, moisture sensors, pH sensors, and combinations thereof.

10. The moldable warming device of claim 1, further comprising:
a vibration modulation controller, wherein the vibration modulation controller controls the stimulation of the first harmonic RFSVG.

11. The moldable warming device of claim 1, further comprising:
a microprocessor; and
a wireless transmitter,
wherein the microprocessor converts analog data generated from the sensor array into a digital data stream, and
wherein the wireless transmitter transmits the digital data stream wirelessly via an antenna.

12. The moldable warming device of claim 11, wherein the wirelessly transmitted digital data stream is configured to be received by a smart device.

13. The moldable warming device of claim 12, wherein the wirelessly received digital data stream is configured to be processed by a smart device application.

14. A method for ophthalmic eyelid therapy, the method comprising the steps of:
applying a moldable warming device to a user's individual eyelid, periorbital, and nasal three-dimensional anatomy and surface topography, the moldable warming device comprising:
a heating disc;
a harmonic resonance frequency stimulation vibration generator (RFSVG);
a coupling device;
a mask, wherein the mask is configured to hold the heating disc, the harmonic RFSVG, and the coupling device for use in parallel utility; and
a sensor array;
generating thermal energy and transferring the thermal energy to an eyelid surface and periorbital structures;
generating harmonic resonance or non-resonance frequency stimulation vibration and transferring the harmonic resonance or non-resonance frequency stimulation vibration over the user's nasal bridge area through the user's nasal bone; and
stimulating nasal sensory nerves to induce tear production reflex in the user's lacrimal function unit.

15. The method of claim 14, wherein the thermal energy is transferred to an inner surface of the eyelid.

16. The method of claim 14, wherein the coupling device is configured to contact the user's eyelid skin for heat transfer.

17. The method of claim 14, wherein the coupling device comprises hydrogel.

18. The method of claim 14, wherein the mask comprises a heat reflective material configured to reflect and direct heat from the heating disc toward the user's eyelid surface.

19. The method of claim 14, wherein the sensor array comprises a thermocouple configured to record temperature of the user's eyelid surface.

20. The method of claim 19, wherein the sensor array includes a flexible printed circuit board where the thermocouple is embedded therein.

21. The method of claim 14, wherein the sensor array comprises sensors selected from the group consisting of: temperature sensors, pressure sensors, moisture sensors, pH sensors, and combinations thereof.

22. The method of claim 14, further comprising the step of:
generating, at the sensor array, a data stream responsive to a vibration and heating profile of the user's individual patient eyelid and periorbital three-dimensional anatomy and surface topography.

23. The method of claim 22, further comprising the steps of:
converting the data stream from analog to digital; and
transmitting the data stream wirelessly via an antenna.

24. The method of claim 23, further comprising the step of:
receiving the wirelessly transmitted data stream using a smart device.

25. The method of claim 24, further comprising the step of:
processing the wirelessly transmitted data stream using a smart device application.

26. A moldable warming device, comprising:
a heating disc;
a first harmonic resonance frequency stimulation vibration generator (RFSVG);
a coupling device; and
a mask configured to hold the heating disc, the first harmonic RFSVG, and the coupling device for use in parallel utility,
wherein the moldable warming device is configured to provide an eyelid surface and periorbital structures with therapeutic warmth,
wherein the first harmonic RFSVG is configured to provide tuned harmonic resonance or non-resonance frequency stimulation vibration over the user's nasal bridge area through the user's nasal bone such that nasal sensory nerves are stimulated to induce tear production reflex in the user's lacrimal function unit.

27. The moldable warming device of claim 26, further comprising:
a second harmonic RFSVG,
wherein the mask is configured to hold the second harmonic RFSVG,
wherein the second harmonic RFSVG is configured to provide the eyelid surface and periorbital structures with tuned harmonic resonance or non-resonance frequency stimulation vibration.

28. A method for ophthalmic eyelid therapy, the method comprising the steps of:
applying a moldable warming device to a user's individual eyelid, periorbital, and nasal three-dimensional anatomy and surface topography, the moldable warming device comprising:
a heating disc;
a first harmonic resonance frequency stimulation vibration generator (RFSVG);
a second harmonic RFSVG;
a coupling device; and
a mask, wherein the mask is configured to hold the heating disc, the first harmonic RFSVG, the second harmonic RFSVG, and the coupling device for use in parallel utility;
reducing viscosity of the user's meibum by generating thermal energy via the heating disc and transferring the thermal energy to the user's eyelid surface and periorbital structures;
mobilizing the user's meibum by generating harmonic resonance or non-resonance frequency stimulation vibration via the first harmonic RFSVG and transferring the harmonic resonance or non-resonance frequency stimulation vibration to the user's eyelid surface and periorbital structures in the absence of transferring the thermal energy to the user's eyelid surface and periorbital structures; and
stimulating the user's nasal sensory nerves to induce tear production reflex in the user's lacrimal function unit by generating the harmonic resonance or non-resonance frequency stimulation vibration via the second harmonic RFSVG and transferring the harmonic resonance or non-resonance frequency stimulation vibration over the user's nasal bridge area through the user's nasal bone.
